(19) United States
(12) Patent Application Publication
(10) Pub. No.: US 2018/0221874 A1
(43) Pub. Date: Aug. 9, 2018

(54) FLUIDIC DEVICES INCORPORATING FUNCTIONAL MUSCLE TISSUE AND METHODS OF USE

(71) Applicant: President and Fellows of Harvard College, Cambridge, MA (US)

(72) Inventors: Kevin Kit Parker, Cambridge, MA (US); Janna C. Nawroth, Boston, MA (US); Ville Kujala, Cambridge, MA (US); Arun R. Shrivats, Cambridge, MA (US)

(73) Assignee: President and Fellows of Harvard College, Cambridge, MA (US)

(21) Appl. No.: 15/750,522

(22) PCT Filed: Aug. 5, 2016
(86) PCT No.: PCT/US2016/045813

§ 371 Date: Feb. 6, 2018

Related U.S. Application Data
(60) Provisional application No. 62/202,213, filed on Aug. 7, 2015.

(51) Int. Cl. B01L 3/00; G01N 33/50

(52) U.S. Cl. CPC B01L 3/502715; G01N 33/5064; B01L 2300/0816; B01L 2300/123; B01L 2300/0645; G01N 33/5061

(57) ABSTRACT

The present invention fluidic devices incorporating functional muscle tissues, methods of making the fluidic devices and methods of use of the fluidic devices.

### RELATED APPLICATIONS

This application is related to U.S. provisional patent application Ser. No. 62/202,213, filed on Aug. 7, 2015, the entire contents of which are incorporated herein by reference in their entirety.

### GOVERNMENT SUPPORT

This invention was made with government support under grant number UH3-TR000522, awarded by the National Institute of Health (NIH); and under grant number W911NF-12-2-0036 awarded by the Defense Advanced Research Projects Agency (DARPA). The government has certain rights in the invention.

### BACKGROUND OF THE INVENTION

Identification and evaluation of new therapeutic agents or identification of suspect disease associated targets typically employ animal models which are expensive, time consuming, require skilled animal-trained staff, and utilize large numbers of animals. In vitro alternatives have relied on the use of conventional cell culture systems which are limited in that they do not allow the three-dimensional interactions that occur between cells and their surrounding tissue. This is a considerable disadvantage as such interactions are well documented as having a significant influence on the growth and activity of cells in vivo because in vivo cells divide and interconnect in the formation of complex biological systems creating structure-function hierarchies that range from the nanometer to meter scales.

Efforts to build biosynthetic materials or engineered tissues that recapitulate these structure-function relationships often fail because of the inability to replicate the in vivo conditions that coax this behavior from ensembles of cells. For example, engineering a functional muscle tissue requires that the sarcomere and myofibrillogenesis be controlled at the micron length scale, while cellular alignment and formation of the continuous tissue require organizational cues over the millimeter to centimeter length scale. Thus, to build a functional biosynthetic material, the biotic-abiotic interface must contain the chemical and mechanical properties that support multiscale coupling.

Accordingly, there is a need in the art for improved methods and systems that are less expensive, time efficient, reproducible, and that permit cell adhesion and tissue morphogenesis in order to recapitulate in vivo structure-function hierarchies for use, e.g., in determining the effect of a test compound on biologically relevant parameters in order to enhance and speed-up the drug discovery and development process.

### SUMMARY

In accordance with some embodiments of the present disclosure, a fluidic device is disclosed. The device includes a porous membrane, a solid support structure, and a flexible substrate. The solid support structure includes a first chamber, a second chamber, and a base. The second chamber is separated from the first chamber by the porous membrane and is in fluid communication with the first chamber via the porous membrane. The base is disposed at the second chamber opposite the porous membrane. The base includes a cyclic olefin copolymer (COC) and a surface. The device further includes a flexible substrate. The flexible substrate includes a polymer layer and/or a hydrogel layer disposed on the surface of the base. The flexible substrate supports growth of a functional muscle tissue.

In some embodiments, a functional muscle tissue is disposed on the flexible substrate.

In some embodiments, a first portion of the surface of the base adjacent to the flexible substrate has a modified surface energy relative to a surface energy of the rest of the surface of the base material to inhibit cell adhesion to the surface of the base.

In some embodiments, the surface energy of the first portion of the surface of the base adjacent to the flexible substrate may be modified by laser etching. In some embodiments, a surface energy of a second portion of the surface of the base underlying the flexible substrate is modified relative to a surface energy of the rest of the surface of the base material to promote adhesion with the flexible substrate. In further embodiments, the surface energy of the second portion of the surface of the base may be modified by oxygen plasma treatment.

In some embodiments, the flexible substrate covers the second portion of the surface of the base and a third portion of the surface of the base. The third portion does not have a modified surface energy to promote adhesion with the flexible substrate. In some embodiments, the flexible substrate is attached to the second portion of the surface of the base material and is not attached to the third portion of the surface of the base.

In some embodiments, the device includes a functional muscle tissue disposed on the flexible substrate. In some embodiments, the functional muscle tissue and the flexible substrate form a functional muscle tissue strip having one or two cantilevered portions.

In some embodiments, a portion or portions of the flexible substrate that are not attached to the surface of the base are configured to deflect away from the surface of the base in response to forces exerted by the functional muscle tissue.

In some embodiments, the flexible substrate has an elongate shape. In a further embodiment, a first end of the flexible substrate and a second end of the flexible substrate opposite the first end are not attached to the surface of the base and a middle portion of the flexible substrate is attached to the second portion of the surface of the base.

In some embodiments, the flexible substrate includes a gelatin layer. In some embodiments, the gelatin layer has an average height in a range of 165 μm to 225 μm.

In some embodiments, a surface of the flexible substrate facing away from the base comprises micro-scale topological features to promote growth of a functional muscle tissue. In a further embodiment, the micro-scale topological features on the surface of the flexible substrate are micromolded features.

In some embodiments, the device further includes a flexible electrode array at least partially disposed between the flexible substrate and the base. In some embodiments, the flexible electrode array is bonded to the surface of the base. In some embodiments, the flexible substrate adheres to the flexible electrode array and to the surface of the base.

In some embodiments, the flexible substrate includes gelatin. In some embodiments, the flexible substrate has an average height in a range of 55 μm to 115 μm. In a further embodiment, the flexible substrate has an average height in a range of 75 μm to 95 μm.

In some embodiments, a surface of the flexible substrate facing away from the base includes micro-scale topological features to promote growth of a functional muscle tissue. In a further embodiment, the micro-scale topological features on the surface of the flexible substrate are micromolded features.

In some embodiments, the device includes a second flexible substrate. The second flexible substrate includes a polymer layer and/or a hydrogel layer disposed on the surface of the base. The second flexible substrate is configured to support growth of a functional muscle tissue. The second flexible substrate is spaced from the flexible substrate by at least 1.5 mm.

In some embodiments, the porous membrane and at least a portion of the first chamber define a first fluid channel. The porous membrane and at least a portion of the second chamber define a second fluid channel. In some embodiments, the porous membrane has a proximal (upstream) end and the surface of the base has a leading portion between the proximal end of the porous membrane and the portion of the surface of the base covered by the flexible substrate. In further embodiments, a length of the leading portion is selected to achieve sufficient uniformity in a drug concentration profile across the flexible substrate for a drug flowing through the first fluid channel at a first rate and diffusing through the porous membrane into a liquid flowing through the second fluid channel at a second rate. The sufficient uniformity is a difference in a drug concentration of less than 50% between an upstream end and a downstream end of the flexible substrate.

In some embodiments, a length of the leading portion is at least 4 mm. In some embodiments, a porosity of the porous membrane is between 5% and 11%. In further embodiments, a porosity of the porous membrane is between 6% and 9%.

In some embodiments, the device includes a growth promoting layer disposed at least partially on the porous membrane in the first chamber. The growth promoting layer is configured to promote adhesion and growth of cells. In some embodiments, the devices further includes a plurality of cells adhered to the growth promoting layer and disposed in the first chamber. In some embodiments, the plurality of cells are selected from the group consisting of epithelial cells, endothelial cells, sensory transducer cells, neuronal cells, hormone-secreting/endocrine cells, glial cells and/or adipocytes.

In some embodiments, the porous membrane and at least a portion of the first chamber define a first fluid channel having a surface opposite the porous membrane. the device further includes a first electrode, a second electrode, and a growth promoting layer. The first electrode is disposed in the first fluid channel at least partially overlying the porous membrane. The second electrode is disposed on the surface of the first fluid channel opposite the first electrode. The growth promoting layer is disposed in the first fluid channel overlying at least a portion of the first electrode and overlying at least a portion of the porous membrane. The growth promoting layer is configured to promote adhesion and growth of epithelial cells, endothelial cells, sensory transducer cells, neuronal cells, hormone-secreting/endocrine cells, glial cells and/or adipocytes.

In some embodiments, the first fluid channel has a proximal end defined near an inflow portion of the first fluid channel and a distal end defined near an outflow portion of the first fluid channel. The first electrode and the second electrode are disposed at the proximal end or at the distal end of the first fluid channel. In further embodiments, the first electrode and the second electrode are disposed away from the flexible substrate. In some embodiments, the first electrode and the second electrode comprise gold. In some embodiments, the first electrode has a thickness in a range of 20 nm to 400 nm. In further embodiments, the first electrode has a thickness in an range of 20 nm to 200 nm.

In some embodiments, each of the first electrode and the second electrode include an adhesion layer including titanium and an overlying layer comprising gold. In some embodiments, the adhesion layer has a thickness in a range of 3 nm and 10 nm.

In some embodiments, the device further includes a third electrode disposed in the first fluid channel at least partially overlying the porous membrane and a fourth electrode disposed on the surface of the first fluid channel opposite the third electrode.

In some embodiments, the porous membrane comprises polycarbonate.

In accordance with embodiments of the present disclosure, a fluidic device is disclosed. The device includes a porous membrane. The device further includes a first channel defining member disposed on the porous membrane. The porous membrane and the first channel defining member define a first fluidic channel. The device further includes a support member providing mechanical support for the fluidic device. The device further includes a base disposed on the support member. The device further includes a second channel defining member disposed on the base. The porous membrane is disposed on the second channel defining member. The device further includes a gasket disposed between the base and the second channel defining member. The base, the second channel defining member, the gasket, and the porous membrane define a second fluidic channel. The device further includes a flexible substrate. The flexible substrate includes a polymer layer and/or a hydrogel layer disposed at least partially on the surface of the base. The flexible substrate is configured to support growth of a functional muscle tissue. The device further includes one or more securing elements that releasably secure the first channel defining member, the porous membrane, the second channel defining member and the base to the support member.

In some embodiments, the fluid device is configured to be disassembled into a first portion including the first channel defining member and the porous membrane and a second portion including the base and the support member.

In some embodiments, the device further includes a growth promoting layer disposed on the porous membrane within the first fluidic channel. The growth promoting layer is configured to promote adhesion and growth of cells.

In some embodiments, the base comprises a cyclic olefin copolymer (COC).

In some embodiments, the device includes a flexible electrode array at least partially disposed between the substrate and the base. In further embodiments, the device further includes a functional muscle tissue disposed on the flexible substrate. In some embodiments, the functional muscle tissue and the flexible substrate form a functional muscle tissue strip having one or two cantilevered portions.

In some embodiments, the functional muscle tissue includes cells selected from the group consisting of cardiac muscle cells, ventricular cardiac muscle cells, atrial cardiac muscle cells, striated muscle cells, smooth muscle cells, vascular smooth muscle cells and combinations thereof.

In accordance with embodiments of the present disclosure, a kit is disclosed. The kit includes any of the devices described herein and a cell seeding well. The well includes a well body having a first surface and a second surface. The well body defines an aperture extending from the first surface to the second surface. The shape of the aperture at the second surface corresponds to a shape of the flexible substrate of the fluidic device. In some embodiments, the aperture tapers from a first cross-sectional area at the first surface to a smaller second cross-sectional area at the second surface.

In accordance with embodiments of the present disclosure, a method is disclosed. The method includes providing a fluidic device. The device further includes a functional muscle tissue disposed on the flexible substrate, a growth promoting layer disposed on the porous membrane, and a plurality of epithelial cells, endothelial cells, sensory transducer cells, neuronal cells, hormone-secreting/endocrine cells, glial cells and/or adipocytes disposed on the growth promoting layer.

In accordance with embodiments of the present disclosure, a method is disclosed. The method includes providing a fluidic device with the first portion separated from the second portion. The method further includes seeding a plurality of muscle cells onto the flexible substrate of the second portion of the fluidic device. The method further includes culturing the plurality of muscle cells to form a functional muscle tissue. The method further includes seeding a plurality of epithelial cells, endothelial cells, sensory transducer cells, neuronal cells, hormone-secreting/endocrine cells, glial cells and/or adipocytes onto the growth promoting layer of the first portion of the fluidic device. The method further includes culturing the plurality of cells on the growth promoting layer. The method further includes assembling the fluidic device thereby forming the first fluidic channel and the second fluidic channel.

In some embodiments, assembling the fluidic device includes positioning the first portion in contact with the second portion. Assembling the fluidic device further includes securing the first portion to the second portion using the one or more securing elements.

In some embodiments, the method includes determining an electrical property of the epithelial cells, endothelial cells, sensory transducer cells, neuronal cells, hormone-secreting/endocrine cells, glial cells and/or adipocytes and determining a contractile function of the functional muscle tissue. In some embodiments, the contractile function is a biomechanical activity. In further embodiments, the biomechanical activity is selected from the group consisting of contractility, cell stress, cell swelling, and rigidity. In some embodiments, the contractile function is an electrophysiological activity. In further embodiments, the electrophysiological activity is a voltage parameter selected from the group including action potential, action potential duration (APD), conduction velocity (CV), refractory period, wavelength, restitution, bradycardia, tachycardia, and reentrant arrhythmia. In some embodiments, the electrophysiological activity is a calcium flux parameter selected from the group consisting of intracellular calcium transient, transient amplitude, rise time (contraction), decay time (relaxation), total area under the transient (force), restitution, focal and spontaneous calcium release.

In some embodiments, the method includes applying a stimulus.

In accordance with embodiments of the present disclosure, a method for identifying a compound that modulates a contractile function of a functional muscle tissue is disclosed. The method includes providing a fluidic device as described herein. The fluidic device further includes a functional muscle tissue disposed on the flexible substrate, a growth promoting layer disposed on the porous membrane, and a plurality of epithelial cells, endothelial cells, sensory transducer cells, neuronal cells, hormone-secreting/endocrine cells, glial cells and/or adipocytes disposed on the growth promoting layer. The method further includes determining the effect of a test compound on a contractile function of the functional muscle tissue in the presence and absence of the test compound. A modulation of the contractile function of the functional muscle tissue in the presence of said test compound as compared to the contractile function in the absence of the test compound indicates that the test compound modulates a contractile function of a functional muscle tissue, thereby identifying a compound that modulates a contractile function of a functional muscle tissue.

In accordance with embodiments of the present disclosure, a method for identifying a compound useful for treating or preventing a muscle disease is disclosed. The method includes providing a fluidic device as described above. The fluidic device further includes a functional muscle tissue disposed on the flexible substrate, a growth promoting layer disposed on the porous membrane, and a plurality of epithelial cells, endothelial cells, sensory transducer cells, neuronal cells, hormone-secreting/endocrine cells, glial cells and/or adipocytes disposed on the growth promoting layer. The method further includes contacting the functional muscle tissue with a test compound. The method further includes determining the effect of the test compound on a contractile function of the functional muscle tissue in the presence and absence of the test compound. A modulation of the contractile function of the functional muscle tissue in the presence of said test compound as compared to the contractile function in the absence of said test compound indicates that the test compound modulates a contractile function the functional muscle tissue, thereby identifying a compound useful for treating or preventing a muscle disease.

In accordance with embodiments of the present disclosure, a fluidic device is disclosed. The device includes a solid support structure having a first chamber and a second chamber operably connected to the first chamber via a porous membrane. At least a portion of the first chamber and the porous membrane defines a fluid channel having a surface opposite the porous membrane. The device further includes a first electrode disposed in the fluid channel at least partially overlying the porous membrane. The device further includes a second electrode disposed on the surface of the fluid channel opposite the first electrode. The device further includes a growth promoting layer disposed in the fluid channel overlying at least a portion of the first electrode and overlying at least a portion of the porous membrane. The growth promoting layer is configured to promote adhesion and growth of epithelial cells, endothelial cells, sensory transducer cells, neuronal cells, hormone-secreting/endocrine cells, glial cells and/or adipocytes.

In some embodiments, the fluid channel has a proximal end defined near an inflow portion of the fluid channel and a distal end defined near an outflow portion of the fluid channel. The first electrode and the second electrode are disposed at the proximal end or at the distal end of the fluid channel.

In some embodiments, the second chamber contains muscle cells.

In some embodiments, the first electrode and the second electrode are disposed away from the muscle cells. In some embodiments, the first electrode and the second electrode comprise gold. In some embodiments, the first electrode has a thickness between 20 nm to 400 nm. In further embodiments, the first electrode has a thickness between 20 nm to 200 nm. In some embodiments, each of the first electrode and the second electrode include an adhesion layer including titanium and an overlying layer comprising gold. In some embodiments, the adhesion layer has a thickness between 3 nm and 10 nm.

In some embodiments, the device includes a third electrode disposed in the fluid channel at least partially overlying the porous membrane. The device further includes a fourth electrode disposed on the surface of the fluid channel opposite the third electrode.

In some embodiments, the device includes endothelial cells cultured on the growth promoting layer.

In some embodiments, the device includes a plurality of cantilevered functional muscle tissue strips disposed in the second chamber.

In accordance with embodiments of the present disclosure, a method of producing a system for determining an electrical property of epithelial cells, endothelial cells, sensory transducer cells, neuronal cells, hormone-secreting/endocrine cells, glial cells and/or adipocytes and determining a muscle tissue function of a functional muscle tissue is disclosed. The method includes providing a fluidic device as previously described. The device further includes a plurality of cantilevered functional muscle tissue strips disposed in the second chamber. The method further includes culturing a layer of epithelial cells, endothelial cells, sensory transducer cells, neuronal cells, hormone-secreting/endocrine cells, glial cells and/or adipocytes on the growth promoting layer.

In accordance with embodiments of the present disclosure, a method for measuring impedance of epithelial cells, endothelial cells, sensory transducer cells, neuronal cells, hormone-secreting/endocrine cells, glial cells and/or adipocytes in a fluidic device is disclosed. The method includes providing a fluidic device as described above. The method further includes providing data regarding a measured baseline frequency-dependent electrical impedance across the fluid channel of the device. The method further includes culturing a layer of endothelial and/or epithelial cells on the growth promoting layer. The method further includes stimulating the fluidic device with an electrical current. The method further includes measuring electrical data from the first, second, third, and fourth electrodes. The method further includes calculating impedance caused by the epithelial cells, endothelial cells, sensory transducer cells, neuronal cells, hormone-secreting/endocrine cells, glial cells and/or adipocytes by subtracting the measured baseline frequency-dependent electrical impedance across the fluid channel from the measured electrical data.

In some embodiments, measuring impedance data includes measuring current via the first and third electrodes, and measuring voltage via the second and fourth electrodes.

In some embodiments, the method includes providing a plurality of cardiomyocyte muscle thin films in the second chamber of the fluidic device.

In some embodiments, providing data regarding the measured baseline frequency-dependent electrical impedance across the fluid channel of the device includes measuring electrical data from the first, second, third, and fourth electrodes prior to culturing the layer of endothelial cells on the growth promoting layer to obtain the measured frequency-dependent baseline electrical impedance across the fluid channel for the fluidic device.

In some embodiments, the fluidic device is simulated with an alternating current of 10 μA.

In accordance with embodiments of the present disclosure, a method of making a fluidic device is disclosed. The method includes providing a base material having a surface with the surface including an area on which a flexible substrate will be formed, a first area adjacent to the area on which the flexible substrate will be formed and a second area within the area on which the flexible substrate will be formed. The method also includes modifying a surface energy of the first area of the surface of the base material relative to a surface energy of a reminder of the surface of the base material to inhibit cell adhesion to the surface of the base in the first area. The method also includes modifying a surface energy of the second area of the surface of the base material relative to a surface energy of a remainder of the surface of the base material to promote bonding between the base and the flexible substrate. The method includes forming the flexible substrate on the surface of the base and providing a solid support structure having one or more chambers in which the base and the flexible substrate are disposed. In some embodiments, the base includes a cyclic olefin copolymer and the flexible substrate includes gelatin. In some embodiments, the surface energy of the first area is modified by laser etching. In some embodiments, the surface energy of the second area is modified by oxygen plasma treatment. In some embodiments, the method further includes culturing functional muscle tissue on the flexible substrate. In some embodiments, the area on which the flexible substrate will be formed includes the second area and a third area in which the surface energy is not modified to promote bonding between the base and the flexible substrate. In some embodiments, the method also includes culturing a functional muscle tissue on the flexible substrate to form a muscle tissue strip including one or more cantilever portions unattached to the base without manual peeling of the flexible substrate.

Additional features and advantages are realized through the techniques of the present disclosure. Other embodiments and aspects of the disclosure are described in detail herein and are considered part of the invention. The recitation herein of desirable objects, which are met by various embodiments of the present disclosure, is not meant to imply or suggest that any or all of these objects are present as essential features, either individually or collectively, in the most general embodiment of the present disclosure, or in any of its more specific embodiments.

### BRIEF DESCRIPTION OF THE DRAWINGS

The features and advantages of the present disclosure will be more fully understood from the following description of exemplary embodiments when read together with the accompanying drawings. The drawings are intended to illustrate the teachings taught herein and are not intended to show relative sizes and dimensions, or to limit the scope of examples or embodiments. In the drawings, the same numbers are used throughout the drawings to reference like features and components of like function.

FIG. 1 schematically depicts a cross-sectional view of a fluidic device taken across a direction of flow according to an embodiment.

FIG. 2 schematically depicts a cross-sectional view of a fluidic device taken along a direction of flow according to an embodiment.

FIG. 3 schematically depicts a top view of a surface of a base having a portion with a modified surface energy to inhibit cell attachment and a portion with a modified surface energy to promote bonding with a flexible substrate according to an embodiment.

FIG. 4 schematically depicts a top view of a surface of a base having a portion with a modified surface energy to inhibit cell attachment and a portion with a modified surface energy to promote bonding with only a portion of a flexible substrate according to an embodiment.

FIG. 5 depicts an exploded perspective view of a fluidic device according to an embodiment.

FIG. 6 is an image of a top view of the fluidic device of FIG. 5 as assembled with a flexible substrate having multiple cantilever portions according to an embodiment.

FIG. 7 is an exploded view of a fluidic device including a flexible electrode array with a first portion of the fluidic device separated from a second portion of the fluidic device according to an embodiment.

FIG. 8 is an image of the fluidic device of FIG. 7 as assembled according to an embodiment.

FIG. 9 is perspective view of the second portion of the fluidic device of FIGS. 7 and 8 showing the gasket in relation to the flexible electrode array according to an embodiment.

FIG. 10 schematically depicts a cross-sectional view taken along a flow direction of a fluidic device including a surface of the base having a leading portion upstream of the flexible substrate that is configured to facilitate more uniform delivery of a drug through the porous membrane to a functional muscle tissue on the flexible substrate according to an embodiment.

FIG. 11 schematically depicts a cross-sectional view taken across a direction of flow of a fluidic device including electrodes to measure electrical properties of cells disposed on a porous membrane in accordance with an embodiment.

FIG. 12 schematically depicts a cross-sectional view taken along a direction of flow of a fluidic device of FIG. 11.

FIG. 13 is an image and a detail of a fluidic device including electrodes to measure electrical properties of cells disposed on a porous membrane in accordance with an embodiment.

FIG. 14 includes images of the fluidic device of FIG. 13 prior to assembly (A), as assembled (B), as assembled and mounted to a carrier (C), and during use (D).

FIG. 15 depicts a perspective view of a cell seeding well according to an embodiment.

FIG. 16 is an image of a batch of gaskets to be used with cell seeding wells according to an embodiment.

FIG. 17A is an image of a cell seeding well mounted on a second portion of a fluidic device including a flexible substrate, a base and a support member according to an embodiment.

FIG. 17B is an image of another view of the cell seeding well of FIG. 17A.

FIG. 18A schematically depicts a seeding well of a cell seeding system in according to an embodiment.

FIG. 18B schematically depicts a ring to hold media for the cell seeding system according to an embodiment.

FIG. 18C is an image of the cell seeding well affixed to the ring of the cell seeding system.

FIG. 18D is an image of a gasket of the cell seeding system according to an embodiment.

FIG. 18E is an image of the cell seeding system mounted to a second portion of a fluidic device being used for cell seeding according to an embodiment.

FIG. 19 depicts an overview of a process for making a flexible substrate on a base with a flexible electrode array probe disposed between the flexible substrate and the base according to an embodiment.

FIG. 20 is an image of a micropatterned surface of a flexible substrate made using the process depicted in FIG. 19.

FIG. 21 is an image of functional muscle tissue grown on the flexible substrate made using the process depicted in FIG. 19.

FIG. 22 is an overview of a process of making a flexible substrate on a base with the flexible substrate having two cantilevers unattached to a surface of the base according to some embodiments.

FIG. 23 is an image of a flexible substrate having cantilever portions made using the process of FIG. 22.

FIG. 24 is a detail view of a corner of a flexible substrate made using the process of FIG. 22 and the underlying base showing where the base was laser etched around the flexible substrate in accordance with an embodiment.

FIG. 25A is a view of an end of a flexible substrate made using the process of FIG. 22 with a functional muscle tissue disposed on the flexible substrate forming a muscle tissue strip with the functional muscle tissue in an uncontracted state.

FIG. 25B is a view of the same end of the muscle tissue strip with the functional muscle tissue in a contracted state causing the end of the muscle tissue strip to deflect away from the underlying base.

FIG. 26 is an image of a system for recording electrical data during seeding and culturing of cells on a flexible substrate using a cell seeding well attached to a second portion of a fluidic device including a flexible electrode array according to some embodiments.

FIG. 27 is a graph of measured cardiac field potentials as a function of time detected by the microelectrode array of the system of FIG. 26 for various concentrations of applied isoproterenol.

FIG. 28 is a graph of QT intervals as a function of applied isoproterenol dose for the data in FIG. 27 showing QT shortening with increased doses consistent with predictions.

FIG. 29 is a graph of measure FITC-Inulin transport across a permeable membrane having an endothelial cell layer as a function of time after cell seeding in a fluidic device including a first channel and a second channel separated by a permeable membrane. The graph indicates development of the endothelial cells into a confluent layer of cells.

FIG. 30A schematically depicts a first design of a fluidic device having a first channel and a second channel separated by a porous membrane with an endothelial layer according to an embodiment.

FIG. 30B depicts results of simulation of diffusion of a drug from the first channel through the endothelial cell layer and the porous membrane and into the second channel for fluidic device of FIG. 30A.

FIG. 31A schematically depicts a second design of a fluidic device having a first channel and a second channel separated by a porous membrane with an endothelial layer according to an embodiment.

FIG. 31B depicts results of simulation of diffusion of a drug from the first channel through the endothelial cell layer and the porous membrane and into the second channel for fluidic device of FIG. 31A.

FIG. 32 schematically depicts an experimental setup for measuring electrical properties across a fluidic channel using the devices depicted in FIGS. 11 through 14.

FIG. 33 includes graphs of baseline measurements of impedance across a channel as a function of frequency for various individual fluidic devices prior to cell seeding.

### DETAILED DESCRIPTION

Described herein are fluidic devices, methods of producing the fluidic devices, and methods of use of the fluidic devices.

In some embodiments, the fluidic devices include a porous membrane, a solid support structure, and a flexible substrate configured to support growth of a functional muscle tissue. The solid support structure includes a first chamber, a second chamber separated from the first chamber by a porous membrane and a base disposed at or in the second chamber opposite the porous member. The base includes a cyclic olefin copolymer (COC) and has a surface on which the flexible substrate is disposed. The base including a COC may be advantageous because COCs are chemically resistant to organic solvents, highly biocompatible, easily cut and machined with lasers and a mill, and have low autofluorescence. As described below, a surface energy of the COC base over one or more selected areas of the base may be modified to inhibit adhesion of cells to the base and in other selected areas may be modified to enhance bonding between the flexible substrate and the base. For example, laser etching may be employed to modify a surface energy of part of the base to inhibit cell attachment. As another example, in embodiments in which the flexible substrate comprises a gelatin, a portion of the surface of the base may be modified with an oxygen plasma treatment to enhance bonding of part or all of the gelatin flexible substrate with the COC base. Modification of the surface energy of the base to promote bonding may also promote bonding with other elements that may be included in the fluidic device, such as a flexible microelectrode array (MEA) disposed at least partially between the flexible substrate and the base in some embodiments. In some embodiments in which only a portion of the flexible substrate is to be attached to the underlying base such that the flexible substrate or a muscle tissue strip formed of the flexible substrate and a functional muscle tissue has cantilevered portions, modification of the surface energy of the underlying base may facilitate production of the device without manual peeling or after cell seeding of cantilever portions of the flexible substrate or the muscle tissue strip.

In some embodiments, the fluidic devices include a porous membrane, a first channel defining member disposed on the porous membrane, a support member that provides mechanical support for the fluidic device, a base disposed on the support member, a second channel defining member disposed on the base, a gasket, a flexible substrate configured to support growth of a functional muscle tissue, and one or more securing elements that releasably secure the first channel defining member, the porous membrane, the second channel defining member and the base to the support member. The modular nature of some fluidic devices described herein is convenient for seeding and growing functional muscle tissue on the flexible substrate with the fluidic device partially disassembled and then easily completing assembly of the fluidic device after the functional muscle tissue is grown. In embodiments that include a growth supporting layer configured to support cells on the porous membrane, the modular nature may be particularly advantageous if the cells be seeded and grown on the growth supporting layer on the porous membrane require different culturing conditions than those grown on the flexible membrane. The modular nature enables separate culturing of cells on the growth supporting layer and cells on the flexible substrate, and then easy assembly of the portions including the cultured cells into the fluidic device. In some embodiments, the modular nature enables electrical measurements of the cells on the flexible substrate during seeding and culturing to assess development of the functional muscle tissue prior to assembly of the full fluidic device.

In some embodiments fluidic devices include a solid support structure having a first chamber and a second chamber operably connected to the first chamber via a porous membrane. At least a portion of the first chamber and the porous membrane define a fluid channel having a surface opposite the porous membrane. The devices also include a first electrode disposed in the fluid channel at least partially overlying the porous membrane and a second electrode disposed on a surface of the fluid channel opposite the first electrode. The devices also include a growth promoting layer disposed in the fluid channel overlying at least a portion of the first electrode and overlying at least a portion of the porous membrane. The growth promoting layer is configured to promote adhesion of cells such as epithelial cells, endothelial cells, sensory transducer cells, neuronal cells, hormone-secreting/endocrine cells, glial cells and adipocytes. The first and second electrodes on opposing surfaces of the fluid channel provide quantitative data regarding changes in electrical properties of cells attached to the porous substrate.

Fluidic devices in accordance with various embodiments and method of using the fluidic devices are described in further detail below.

### Devices of the Invention

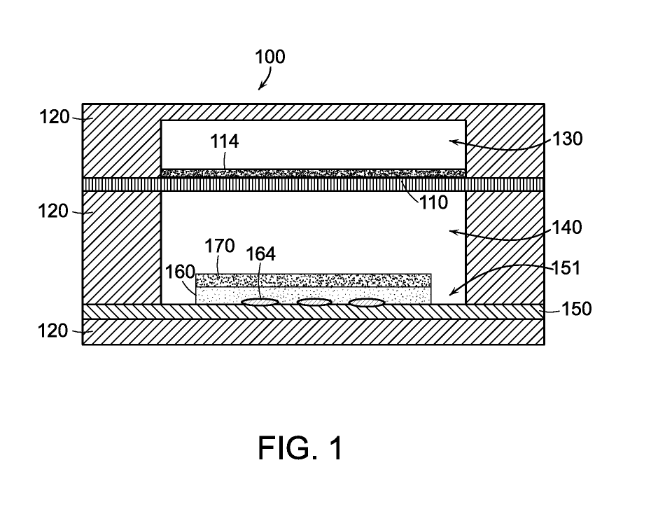

FIG. 1 schematically depicts a fluidic device 100 in accordance with some embodiments. The fluidic device 100 includes a porous membrane 110, a solid support structure 120, a base 150, and a flexible substrate 160. The solid support structure 120 includes a first chamber 130 and a second chamber 140 separated from the first chamber by the porous membrane 100. The second chamber 140 is in fluid communication with the first chamber 130 via the porous membrane 110. The base 150 is disposed at least partially in the second chamber 140 opposite the porous membrane 110. The base 150 includes a cyclic olefin copolymer (COC).

The flexible substrate 160 includes a polymer layer and/or a hydrogel layer disposed on a surface 151 of the base 150. In some embodiments, the flexible substrate 160 comprises a gelatin layer. Additional and alternative polymers and hydrogels that may be included in the flexible substrate are described below.

Hydrogels that can be included in the flexible substrate include, for example, polyacrylamide gels, poly(N-isopropylacrylamide), pHEMA, collagen, fibrin, gelatin, alginate, and dextran. In one embodiment the hydrogel is alginate. In another embodiment, the hydrogel is gelatin. In one embodiment, the stiffness of the hydrogel is tuned to mimic the mechanical properties of healthy muscle tissue, e.g., cardiac tissue in vivo, e.g., to have a Young's modulus of about 10, 11, 12, 13, 14, 15, 16, 17, 18, 19, or about 20 kPa. In another embodiment, the stiffness of the hydrogel is tuned to mimic the mechanical properties of diseased muscle tissue, e.g., cardiac tissue in vivo, e.g., to have a Young's modulus of greater than about 45, 46, 47, 48, 49, 50, 51, 52, 53, 54, or about 55 kPa.

Examples of the elastomers that can be used to form a polymer layer of the flexible substrate include polydimethylsiloxane (PDMS) and polyurethane. In one embodiment, the PDMS, once cured is opaque (e.g., light-absorbing). In other embodiments, thermoplastic or thermosetting polymers are used to form the flexible polymer layer. Alternative non-degradable polymers include polyurethanes, silicone-urethane copolymers, carbonate-urethane copolymers, polyisoprene, polybutadiene, copolymer of polystyrene and polybutadiene, chloroprene rubber, Polyacrylic rubber (ACM, ABR), Fluoro silicone Rubber (FVMQ), Fluoroelastomers, Perfluoroelastomers, Tetrafluoro ethylene/propylene rubbers (FEPM) and Ethylene vinyl acetate (EVA).

In still other embodiments, biopolymers, such as collagens, elastins, polysaccharides, and other extracellular matrix proteins, are included in the flexible substrate. Suitable biodegradable elastomers include hydrogels, e.g., alginate and gelatin, elastin-like peptides, polyhydroxyalkanoates and poly(glycerol-sebecate). Suitable non-elastomer, biodegradable polymers include polylactic acid, polyglycolic acid, poly lactic glycolic acid copolymers.

In one embodiment, a polymer layer included in the flexible substrate comprises polydimethylsiloxane (PDMS). Thickness of the PDMS layer can be controlled by the viscosity of the prepolymer and by the spin-coating speed (if spin coated), ranging from 14 to 60 μm thick after cure. The viscosity of the prepolymer increases as the cross-link density increases. This change in viscosity between mixing and gelation can be utilized to spin-coat different thicknesses of polymer layers. Alternatively the spin-coating speed can be increased to create thinner polymer layers. After spin-coating, the resulting polymer scaffolds are either fully cured at room temperature (generally, about 22° C.) or at 65° C. In some embodiments, the polymer or hydrogel is deposited and molded, but not spin coated.

In one embodiment, polymeric fibers prepared as described in U.S. Patent Publication No. 2012/0135448, (the entire contents of which are incorporated herein by reference) may be used in the polymer layer for the flexible substrate.

In one embodiment, e.g., nanoparticles and/or fluorescent beads, e.g., fluorospheres, are mixed with the hydrogel prior to cross-linking and/or the flexible polymer layer prior to depositing (e.g., spin coating) the polymer layer onto the base.

The flexible substrate 160 is configured to support growth of a functional muscle tissue 170 disposed on the flexible substrate 160.

In some embodiments, a surface of the flexible substrate 160 facing away from the base 150 includes micro-scale topological features to promote growth of the functional muscle tissue 170. In some embodiments, the micro-scale topological features on the surface of the flexible substrate 160 are micromolded features. In other embodiments, the micro-scale topological features may be optically patterned into the hydrogel, e.g., gelatin, as described in U.S. Provisional Application No. 62/371,385, filed on even date herewith (Attorney Docket No.: 117823-14001), the entire contents of which are incorporated herein by reference). The micro-scale topological features enable long-term culture of aligned cells on the flexible substrate 160.

In some embodiments, the functional muscle tissue comprises cells including cardiac muscle cells, ventricular cardiac muscle cells, atrial cardiac muscle cells, striated muscle cells, smooth muscle cells, vascular smooth muscle cells and combinations thereof.

As used herein, a “functional muscle tissue” refers to a muscle tissue prepared in vitro which displays at least one physical characteristic typical of the muscle tissue in vivo; and/or at least one functional characteristic typical of the muscle tissue in vivo, i.e., is functionally active.

For example, a physical characteristic of a functional muscle tissue may include the presence of parallel (to the long axis of the cells) myofibrils with or without sarcomeres aligned in z-lines, and/or that the myofibrils cross cell-to-cell junctions, and/or that the cells maintain a registered array or sarcomeres, and/or that the cells form cell-to-cell gap junctions and/or cell-to-cell adherens junctions. Methods to determine such physical characteristics include, for example, microscopic analyses, such as, fluorescent microscopy, confocal microscopy, two-photon microscopy, and the like, immunohistochemical analyses, e.g., staining for connexin 43 to determine if the cells have formed electrically-competent junctions, staining for β-catenin to determine if the cells have formed mechanically-competent junctions, staining for β-actin and determining, e.g., the orientational order parameter (OOP) of the networks to determine if the cells have formed registered myofibrils.

A functional characteristic of a functional muscle tissue may include an electrophysiological activity, such as an action potential, or biomechanical activity, such as contraction. For example, the cells of a functional muscle tissue may be mechanically and electrically integrated, e.g., the cells synchronously contract, and/or the cells generate a contractile force, and/or the contractions of the cells are in phase, and/or the contractile force at the medial cell-to-cell junctions of the cells are about the same, and/or the cells exhibit synchronous Ca+2 transients, and/or the cells exhibit substantially the same Ca+2 levels, and/or the cells exhibit peak systolic and/or diastolic forces that are about the same.

Methods to determine such functional characteristics include, for example, microscopic analyses, such as fluorescent microscopy, confocal microscopy, two-photon microscopy, optical detection of deflection of the underlying flexible substrate due to contraction of the tissue and the like, immunohistochemical analyses, e.g., vinculin staining, traction force microscopy, ratiometric Ca+2 imaging, optical mapping of the action potentials.

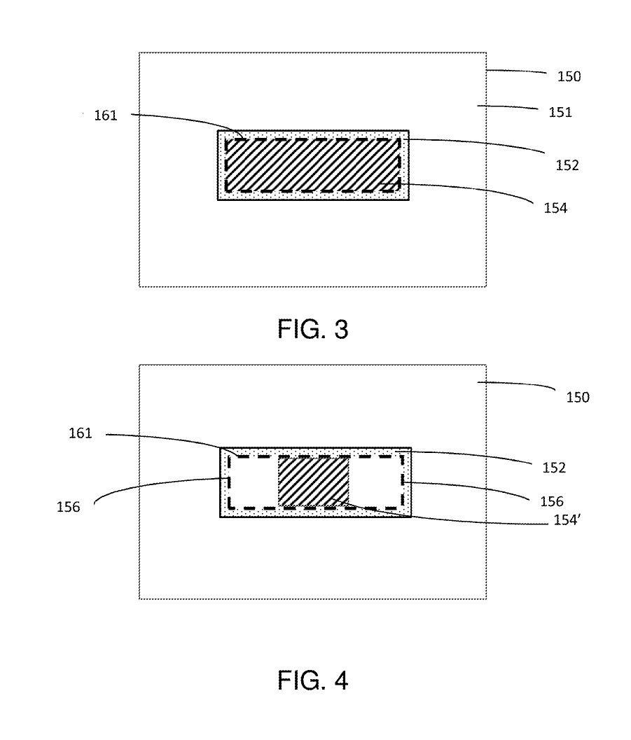

In some embodiments, most or all of the flexible substrate 160 adheres to or is bonded to the surface 151 of the base 150, as shown in FIG. 1. In some embodiments, at least a portion of the surface 151 of the base is modified to promote adhesion or bonding between the flexible substrate 160 and the base 150 as described in more detail below with respect to FIGS. 3 and 4.

In some embodiments the fluidic device 100 includes an electrode array (e.g., a microelectrode array (MEA), a flexible MEA) to measure electrical properties of the functional muscle tissue 170 on the flexible substrate 160. In some embodiments, the fluidic device 100 also includes a flexible electrode array 164 disposed between the flexible substrate 160 and the surface 151 of the base 150. In some embodiments, the flexible electrode array 164 is bonded to the surface 151 of the base 150. In some embodiments, the flexible substrate 160 adheres to the flexible electrode array 164 and to the surface 151 of the base 150. In some embodiments, a surface energy of the surface 151 of the base 150 in selected area may be modified to promote bonding between the flexible electrode layer 164 and the base 150.

A thickness or height of the flexible substrate 160 may be selected such that it provides sufficient height to support the desired micro-scale topological features while remaining sufficiently short/thin to obtain reliable electrical measurements of the functional muscle tissue 170 through the flexible substrate 160 using the flexible electrode array 164. In some embodiments, the flexible substrate 160 includes a gelatin layer having a thickness in a range of about 55 μm to about 115 μm or a range of about 75 μm to about 95 μm.

In some embodiments, the porous membrane 110 is composed of a polycarbonate material. In some embodiments, the porosity of the porous membrane 110 is between 5% and 11%. In further embodiments, the porosity of the porous membrane 110 is between 6% and 9%.

In some embodiments, the fluidic device 100 also includes a growth promoting layer 114 to promote adhesion and growth of cells on the porous membrane 110. The growth promoting layer 114 is disposed at least partially on the porous membrane 110 in the first chamber 130 In some embodiments, the cells grown on the porous membrane include, but are not limited to, epithelial cells, endothelial cells, sensory transducer cells, neuronal cells, hormone-secreting/endocrine cells, glial cells and/or adipocytes. In some embodiments, the cells grown on the porous membrane and the porous membrane 110 act as a vascular-like barrier between chambers of the fluidic device 100, e.g., exposing the muscle tissue in the second chamber to, e.g., O2, CO2, small molecules that can diffuse through the porous membrane and cells thereon.

In some embodiments, the growth promoting layer is a coating on the porous membrane. In some embodiments, the growth promoting layer includes extracellular matrix molecules (ECM), or other proteins such as growth factors or ligands. In some embodiments, the surface of the porous membrane can be activated with any art-recognized reactions, such that ECM molecules, proteins such as growth factors or ligands, can be attached to it.

In some embodiments, the porous membrane is not seeded with cells. In other embodiments, the porous membrane is seeded with cells. In some embodiments where cells are seeded on the porous membrane, cells can be seeded on one side or both sides of the porous membrane. In some embodiments, both sides of the porous membrane can be seeded with the same cell types. In other embodiments, both sides of the porous membrane can be seeded with different cell types.

In some embodiments, the porous membrane can be seeded with at least one layer of cells, including, at least 2 layers of cells or more. Each layer of cells can be the same or different.

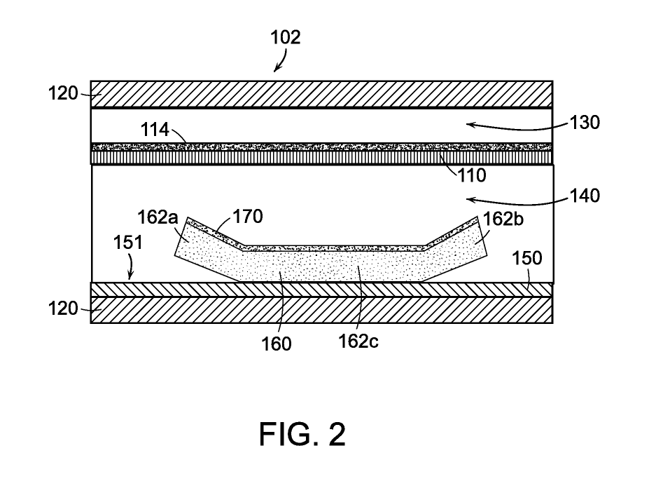

FIG. 2 schematically depicts a fluidic device 102 in accordance with some embodiments. Similar to fluidic device 100 described above, the fluidic device 102 includes a porous membrane 110, a solid support structure 120 having a first chamber 130 and a second chamber 140, a base 150, and a flexible substrate 160. However, as depicted in FIG. 2, in fluidic device 102, one or more portions 162a, 162b of the flexible substrate 160 are not adhered or attached to the surface 151 of the base. Modification of the surface energy of a portion of the surface 161 of the base for attachment of only a portion of the flexible substrate 160 to the underlying surface 151 of the base is described in more detail below with respect to FIG. 4.

A portion or portions 162a, 162b of the flexible substrate 160 that are not attached to the surface 151 of the base 150 are configured to deflect away from the surface of the base 150 in response to forces exerted by a functional muscle tissue 170 on the flexible substrate 160. The deflection of portions of the flexible substrate 160 can be detected or measured (e.g., optically) to obtain measurements of contractile forces exerted on the flexible substrate by the functional muscle tissue. As used herein, a functional muscle tissue on a flexible substrate in which one or more portions of the flexible substrate are not attached to the underlying base and are free to deflect away from a surface of the base in response to contraction of the functional muscle tissue is referred to herein as a muscle tissue strip. In some embodiments, the functional muscle tissue 170 is disposed on the flexible substrate 160 to form a functional muscle tissue strip having one or two cantilevered portions 162a, 162b. In some embodiments, the flexible substrate 160 has an elongate shape and a first end 162a of the flexible substrate and a second end 162b of the flexible substrate opposite the first end are not attached to the surface 151 of the base and a middle portion 162c of the flexible substrate is attached to the surface of the base.

Similar to flexible substrate 160 of fluidic device 100, a surface of the flexible substrate 160 facing away from the 151 of the base includes micro-scale topological features to promote growth of the functional muscle tissue 170. In some embodiments, the flexible substrate 160 comprises gelatin and has an average height in a range of about 165 μm to about 225 μm. The average height or thickness of the flexible substrate 160 may be selected to obtain a desired range of deflections in the one or more cantilevered portions in response to contractile forces exerted by the functional muscle tissue 170 on the flexible substrate 160.

In some embodiments, the fluidic device 100, 102 may include a second flexible substrate (not shown) comprising a polymer layer and/or a hydrogel layer disposed on the surface 151 of the base 150. The second flexible substrate may be configured to support growth of a second functional muscle tissue (not shown). In some embodiments, the second flexible substrate may be spaced from the first flexible substrate by at least about 1.5 mm to prevent cells growing on one flexible substrate from “bridging” the gap with cells growing on the second flexible substrate.

FIGS. 3 and 4 each show a top view of the base 150 having areas of modified surface energy in accordance with some embodiments. Dotted line 161 indicates the area of the surface 151 of the base that would be covered by the flexible substrate 160. In some embodiments, a first portion 152 of the surface 151 of the base adjacent to the flexible substrate 161 has a modified surface energy relative to a surface energy of the rest of the surface 151 of the base 150 material to inhibit cell adhesion to the surface of the base 150 (see dotted area 152 identifying the first portion of the surface of the base). For example, in some embodiments, the surface energy of the first portion 152 of the surface of the base 150 adjacent to the flexible substrate 160 is modified by laser etching. Laser etching changes the surface chemistry of the portion of the base 150 surrounding the flexible substrate 160 to inhibit cell adhesion. In some embodiments, the laser etching is carbon dioxide laser-etching.

In some embodiments, a surface energy of a second portion 154, 154′ of the surface of the base 150 underlying the flexible substrate 160 is modified relative to a surface energy of the rest of the surface of the base 150 material to promote adhesion with or bonding to the flexible substrate 160 (see striped areas 154, 154′ identifying the second portion of the surface of the base). For example, the surface energy of the second portion 154, 154′ of the surface of the base 150 may be modified by oxygen plasma treatment. In some embodiments, the second portion 154 of the surface of the base includes most or all of the area under the flexible substrate (i.e., most or all of the area within dotted line 161) as shown in FIG. 3. Modifying the surface energy of most or all of the area of the base that will be covered by the flexible substrate 160 is particularly useful in embodiments such as that schematically depicted in FIG. 1 in which a flexible electrode array is employed to measure electrical changes in functional muscle tissue disposed on the flexible substrate.

In some embodiments, only some of the area of the surface of the base 151 that will be covered by the flexible substrate is modified to promote adhesion between the base and the flexible substrate. In some embodiments, the area of the base surface 151 covered by the flexible substrate 161 includes the second portion 154′ that has a modified surface energy to promote adhesion and a third portion 156 of the surface of the base that does not have a modified surface energy to promote adhesion with the flexible substrate as shown in FIG. 4. In such an embodiment, the flexible substrate 160 attaches to the second portion 154 of the surface of the base 150 material and but does not attach to the third portion 156 of the surface of the base 150. Using a base having areas of modified surface energy as shown in FIG. 4 for the fluidic device would result in the portions of the flexible substrate that overlay the third portion 156 of the area of the base surface being unattached to the underlying substrate and free to deflect away from the underlying substrate in response to forces exerted by functional muscle tissue as shown in the device of FIG. 2 (see portions 162a, and 162b of the flexible substrate).

Some techniques for forming functional muscle tissue strips require the manual peeling or manual separation of a cantilevered portion of the muscle tissue strip from the underlying layer (e.g., the base) and from cells in the functional muscle tissue that also adhere to the underlying layer (e.g., the base). In embodiments that rely on deflection of portions of the flexible substrate away from the surface of the base, modification of the surface energy of portions of the base to resist cell adhesion (e.g., in first portion 152) and modification of the surface energy of the a portion of the base to promote adhesion over only a portion of the area that will be covered by the flexible substrate (e.g., in second portion 154′, but not third portion 156) both limits cell adhesion and enables free motion of the cantilever portion or portions (162a, 162b) of the flexible substrate without the use of manual peeling. Avoiding manual peeling during manufacture of fluidic devices with flexible substrates having one or more cantilevered portions simplifies the manufacturing process and can reduce errors and potential damage to tissue and/or devices in manufacturing.

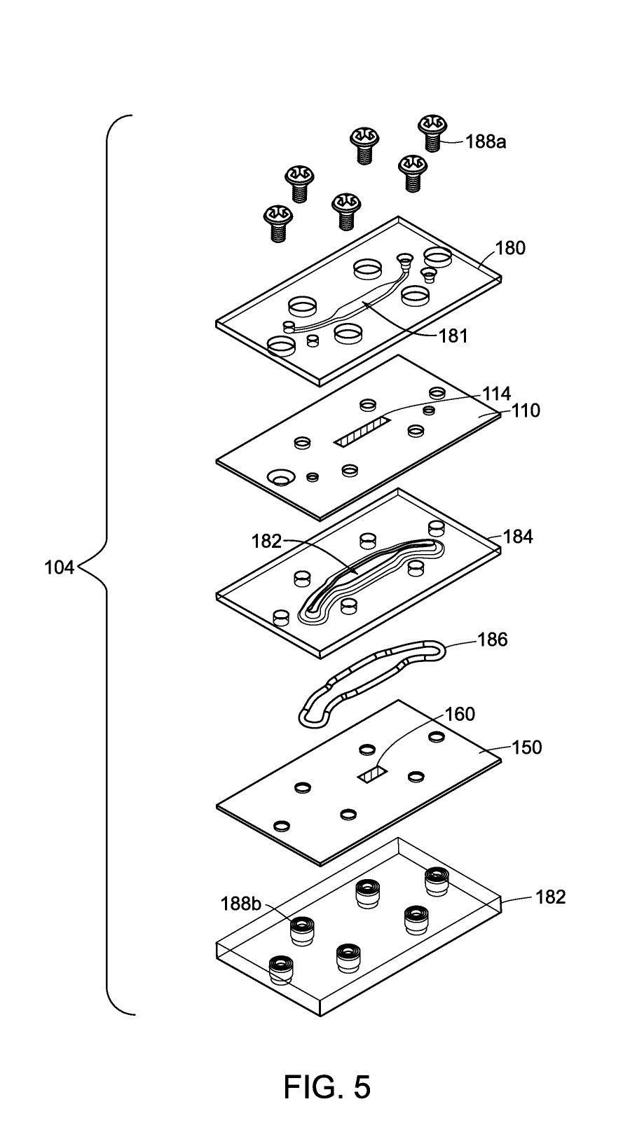

FIG. 5 is an exploded perspective view of elements of a fluidic device 104 having a modular structure according to an embodiment. The fluidic device 104 includes a porous membrane 110 and a first channel defining member 180. When the fluidic device 104 is assembled with the first channel defining member 180 disposed on the porous membrane 110, the porous membrane 110 and the first channel defining member 180 define a first fluidic channel 181. The fluidic device 104 also includes a support member 182 that provides mechanical support for the fluidic device 104 and a base 150 disposed on the support member 182 when the fluidic device 104 is assembled. The fluidic device 104 also includes a second channel defining member 184. In some embodiments, the fluidic device 104 further includes a gasket 186. When the fluidic device 104 is assembled, the second channel defining member 184 is disposed on the base 150, and the porous membrane 110 is disposed on the second channel defining member 184. In embodiments that include a gasket 186, the gasket 186 is disposed between the second channel defining member 184 and the base 150. When the fluidic device 104 is assembled, the base 150, the second channel defining member 184, the gasket 186 (if used), and the porous membrane 110 define a second fluidic channel 182. The fluidic device 104 also include a flexible substrate 160 that is disposed on the base 150. The fluidic device 104 includes one or more securing elements 188 that releasably secure the first channel defining member 180, the porous membrane 110, the second channel defining member 184 and the base 150 to the support member 182. In some embodiments, the fluidic device 104 includes a growth promoting layer 114 disposed on the porous membrane within the first fluidic channel 181.

The flexible substrate 160 includes a polymer layer and/or a hydrogel layer disposed on the surface of the base as described above with respect to FIGS. 1 and 2. The flexible substrate is configured to support growth of a functional muscle tissue as described above.

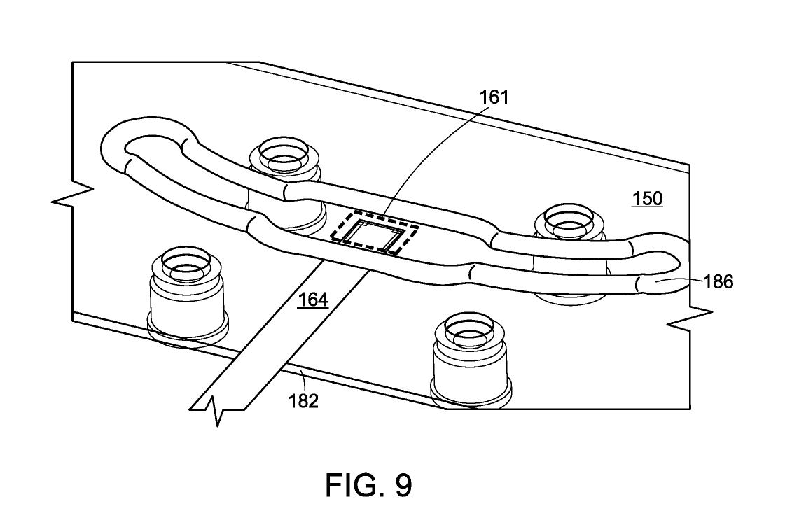

The function of the gasket 180 is discussed in further detail below with respect to FIG. 9. In some embodiments, the gasket 180 is composed of polydimethylsiloxilane (PDMS). The gasket 180 may have various different shapes and is not limited by its depiction in the figures.

The elements of the fluidic device 100 are secured using one or more securing elements 188a, 188b. The securing elements 188a, 188b can be screws, nuts and bolts, snaps, straps, clips, bands, or any other suitable elements for releasably securing the components of the fluidic device 100. In an embodiment, the securing elements are screws 188a and threaded inserts 188b embedded in support member 182.

In the fluidic device 104, the first channel defining member 180, the second channel defining member 184, and the support member 182 are each part of the solid support structure of the fluidic device. In some embodiments, at least portions of the solid support structure 120 are made of polycarbonate material or an acrylic material. When assembled, the first channel defining member 180 and the porous membrane 110 define the first chamber of the fluidic device 104. When assembled, the second channel defining member 184, the porous membrane 110 and the support member 182 define the second chamber of the fluidic device 104. In some embodiments, the base 150 comprises a COC.

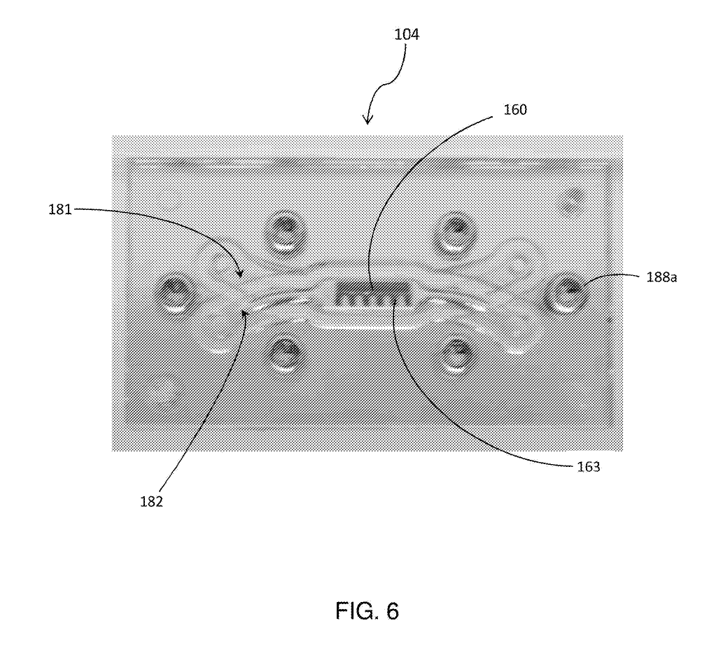

FIG. 6 is top view of the assembled fluid device 104 showing the first channel 181 partially overlaying the second channel 182. In some embodiments, the fluidic device may include a flexible substrate 160 with multiple cantilever portions 163 that are not attached to the underlying base as depicted in FIG. 6.

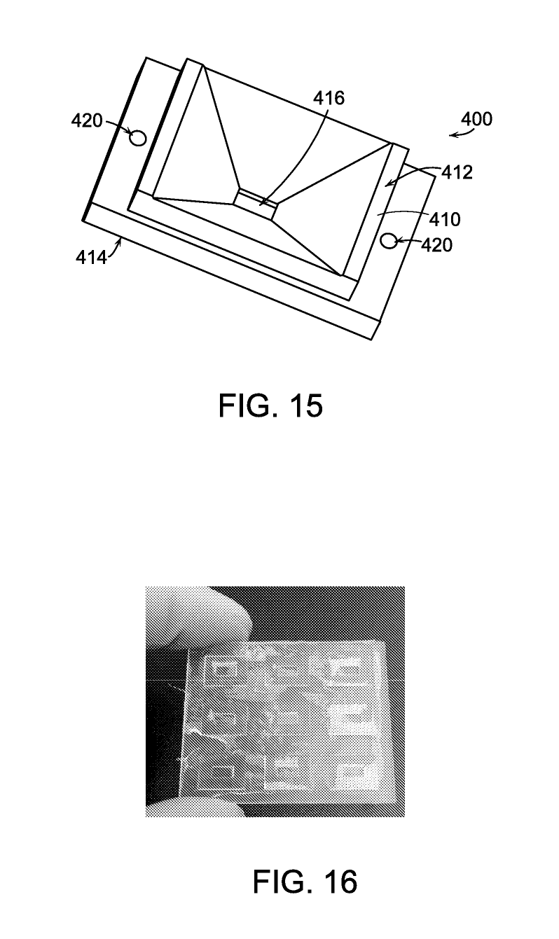

The fluidic device 104 is configured to be easily disassembled into a first portion, which includes the first channel defining member 180 and the porous membrane 110, and a second portion, which includes the base 150 and the support member 120, and then easily reassembled. In some embodiments, the first portion also includes the second channel defining member 184. The securing elements 188a, 188b can be used to secure the first portion to the second portion. In some embodiments, the fluidic device 110 may be provided in a disassembled state with the first portion separate from the second portion. Separating the fluidic device 104 into a first portion and a second portion can facilitate seeding and growth of cells on the flexible substrate 160 and on the growth promoting layer 114 on the porous membrane 110. For example, with the fluidic device separated into two or more portions, the cells on the flexible substrate 160 can be seeded and cultured separately from the cells on the porous membrane 110 and different culturing conditions can be used for each. Additional description of cell seeding and culturing is provided below with respect to FIGS. 15 through 18E and in the methods section.

In some embodiments, the first channel defining member 180 and the porous membrane 110 are bonded to each other (e.g., using an adhesive or another type of permanent or semi-permanent bond). In some embodiments, the porous membrane 110 and the second channel defining member 184 are bonded to each other (e.g., using an adhesive or another type of permanent or semi-permanent bond). In some embodiments, porous membrane 110 is bonded to both the first channel defining member 180 and to the second channel defining member 184 (e.g., via adhesive-free bonding, using an adhesive or another type of permanent or semi-permanent bond). For example, for a porous membrane of polycarbonate and first and second channel defining members of polycarbonate, adhesive-free bonding may be achieved by vaporizing a polycarbonate solvent (e.g., Dichloromethane (DCM)) onto relevant surfaces of the channel defining members, followed by aligning the channel defining members and the porous membranes and bringing them into contact with each other, heating all three to near the glass transition temperature of polycarbonate (T_{g}˜150° C.), and applying a pressure about 135 lbs./in² (931 kPa) for 1 hour. A similar procedure may be employed for adhesive-free bonding of a porous membrane and first and second channel members made from a polymer other than polycarbonate with the solvent, heating temperature and pressure applied adjusted accordingly. For a porous membrane made from a different polymer than that of the first or second channel defining member, an adhesive may be employed.

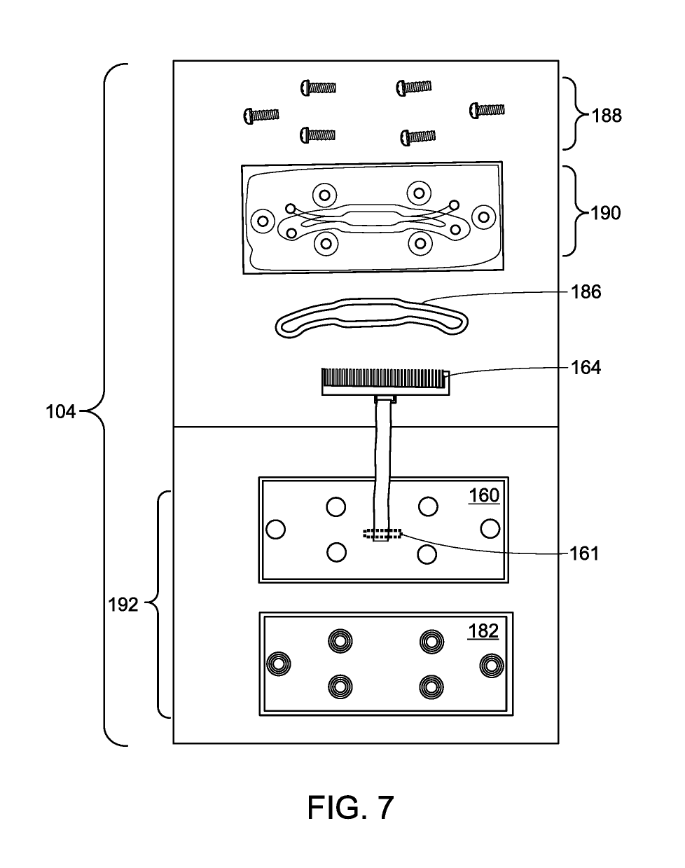

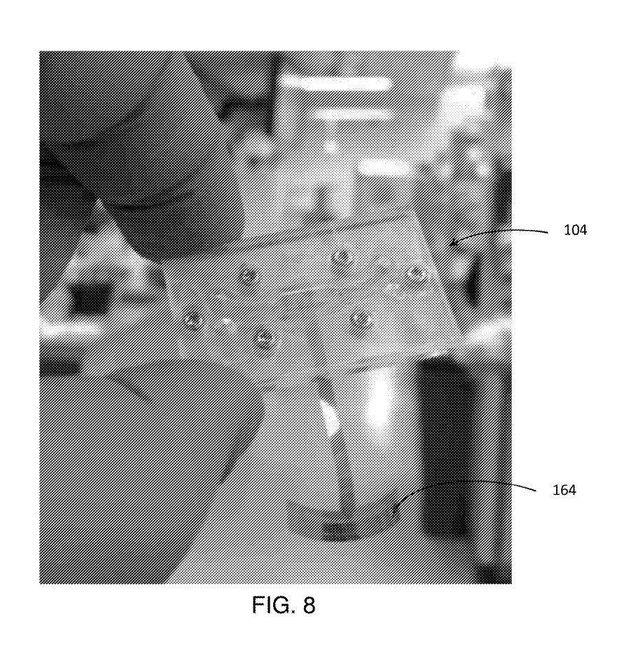

In some embodiments, the fluidic device 104 includes a flexible electrode array between the flexible substrate 160 and the base 150. FIG. 7 is an exploded view of the elements of the fluidic device 104 including a flexible electrode array 164 according to one embodiment. FIG. 8 is an image of the fluidic device 104 in FIG. 7 fully assembled. In FIG. 7, the first channel defining member 180, the porous membrane 110 and the second channel defining member 184 are bonded together to form a first portion 190 of the fluidic device 104. The first portion 190 of the fluidic device is separate from the base 150 and support member 182 of the second portion 192 of the fluidic device. In some embodiments, the flexible electrode array 164 is at least partially disposed between the flexible substrate 160 and the base 150. The flexible substrate is not shown in FIG. 7, however a location for placement of the flexible substrate is shown with dotted line 161. The flexible electrode array 164 may extend from the fluidic device 104 a sufficient distance to contact measurement devices external to the fluidic device 104.

In some embodiments, the flexible electrode array 164 is bonded to the surface of the base 150. In some embodiments, the flexible substrate 161 adheres to the flexible electrode array 164 and the base 150. In these embodiments, the gasket 180 may be employed to aid in hold the flexible electrode array 164 in place. In some embodiments, the flexible electrode array 164 is secured to the base 150 by pressure from the gasket 180. For example, FIG. 9 depicts the flexible electrode array 164 disposed on the base 160 of a fluidic device 104 with the gasket 186 over a portion of the flexible electrode array 164. For ease in visualization, the first portion 190 of the fluidic device is not shown. The first portion of the fluidic device exerts pressure on the gasket 186, which, exerts pressure on the flexible electrode array 164 that aids in securing the flexible electrode array to the base 160. The flexible substrate is not shown in FIG. 9, however a location for placement of the flexible substrate is shown with dotted line 161.

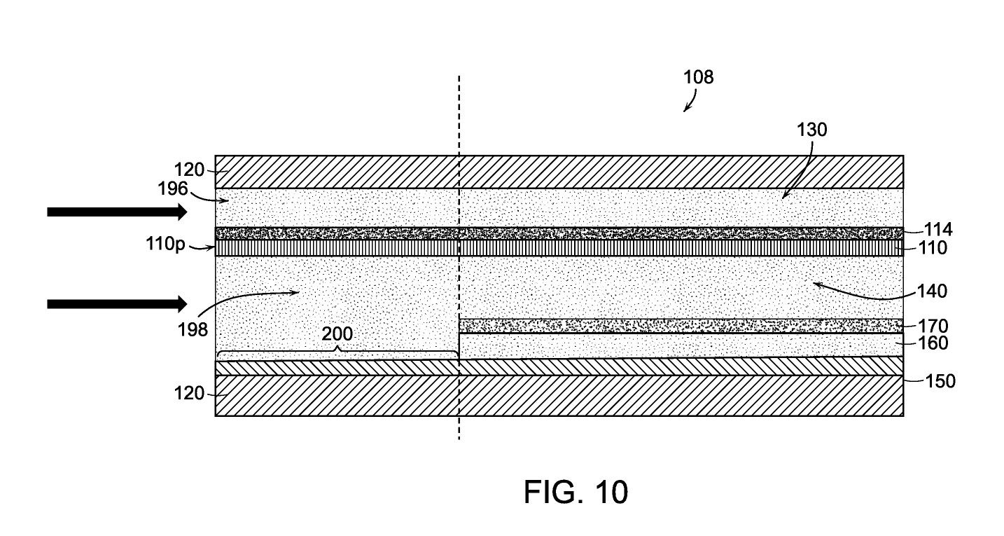

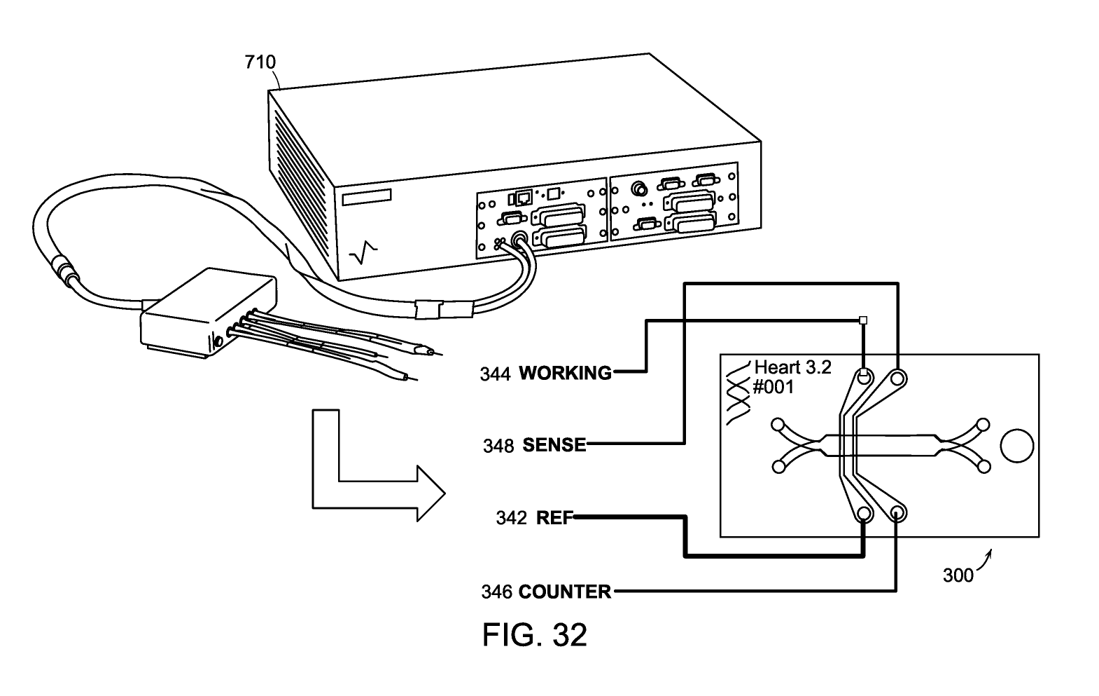

In some embodiments, a fluidic device has a configuration that facilitates achieving a specified level of uniformity of concentration of a drug across a functional muscle tissue. For example, FIG. 10 schematically depicts a cross-sectional view of a fluidic device 108 including a porous membrane 110, a solid support structure 120, a base 150, and a flexible substrate 160. In some embodiments, a functional muscle tissue 170 is disposed on the flexible substrate 160. The solid support structure 120 includes a first chamber 130 and a second chamber 140 separated from the first chamber by the porous membrane 100. The second chamber 140 is in fluid communication with the first chamber 130 via the porous membrane 110. The porous membrane 110 and at least a portion of the first chamber 130 define a first fluid channel 196 as shown. The porous membrane 110 and at least a portion of the second chamber 140 define a second fluid channel 198 as shown with the proximal end 110p of the porous membrane being on the upstream end of the second fluid channel. The surface 151 of the base 150 has a leading portion 200 corresponding the distance along second fluid channel between the proximal end 110p of the porous membrane and the portion of the surface of the base 150 covered by the flexible substrate 160. A length of the leading portion 200 is selected to achieve sufficient uniformity in a drug concentration profile across the flexible substrate 160 for a drug flowing through the first fluid channel 196 at a first rate and diffusing through the porous membrane 150 into a liquid flowing through the second fluid channel 198 at a second rate. In some embodiments, a sufficient uniformity is a difference in a drug concentration of less than 50% between an upstream end and a downstream end of the flexible substrate 160. In an embodiment, the leading portion 192 is at least about 4 mm long and the portion of the base 150 covered by the flexible substrate 160 is at least about 8 mm long. In some embodiment, a length of a leading portion is in a range of about 2 mm to about 6 mm. In some embodiments, a length of a leading portion is in a range of about 3 mm to about 5 mm. In some embodiments, the porosity of the porous membrane 110 is between about 5% and about 11%. In some embodiments, the porosity of the porous membrane 110 is between about 6% and about 9%. Computer simulation of a drug concentration at the flexible substrate for different configuration of a fluid device are described below in the examples with respect to FIG. 31A through 32B.

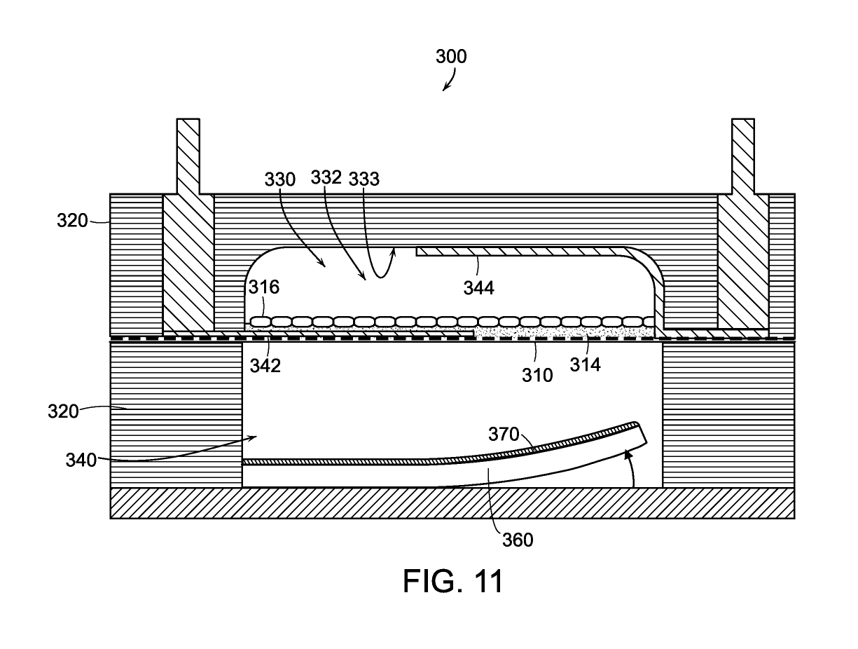

In some embodiments, a fluidic device also includes electrodes that measure electrical properties of cells disposed on the porous membrane (e.g., an impedance of a cell layer on the porous membrane). For example, in some embodiments, a fluidic device also includes a first electrode disposed in the first fluid channel at least partially overlying the porous membrane, a second electrode disposed on a surface of the fluid channel opposite the first electrode, and a growth promoting layer disposed in the first fluid channel overlying at least a portion of the first electrode. Further description of embodiments including electrodes that measure electrical properties of cells disposed on the porous membrane are provided below with respect to FIGS. 11 through 14. Aspects of embodiments depicted in FIGS. 11-14 may be combined with or incorporated into any other fluidic devices disclosed herein.

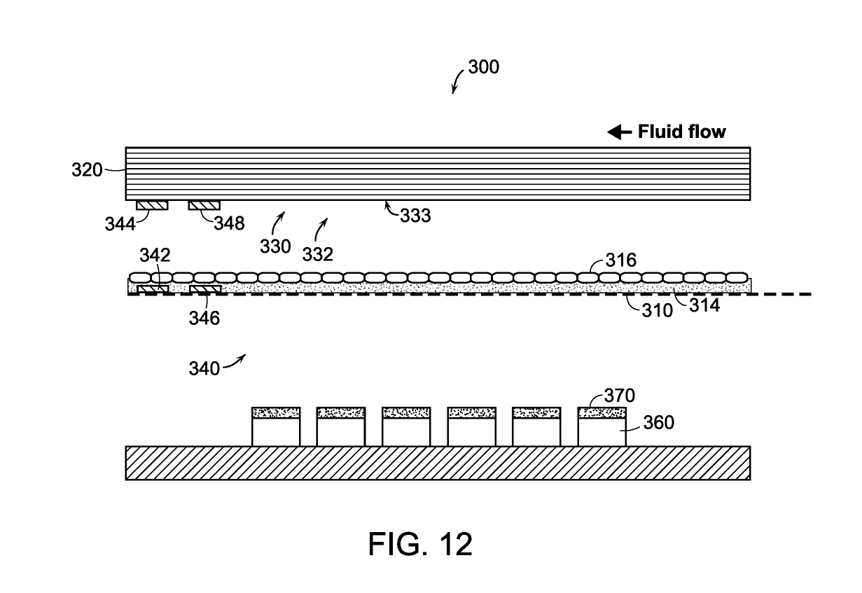

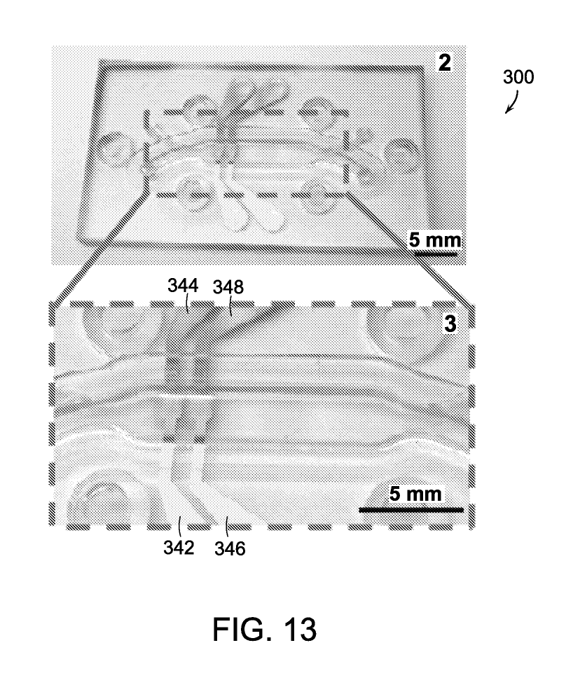

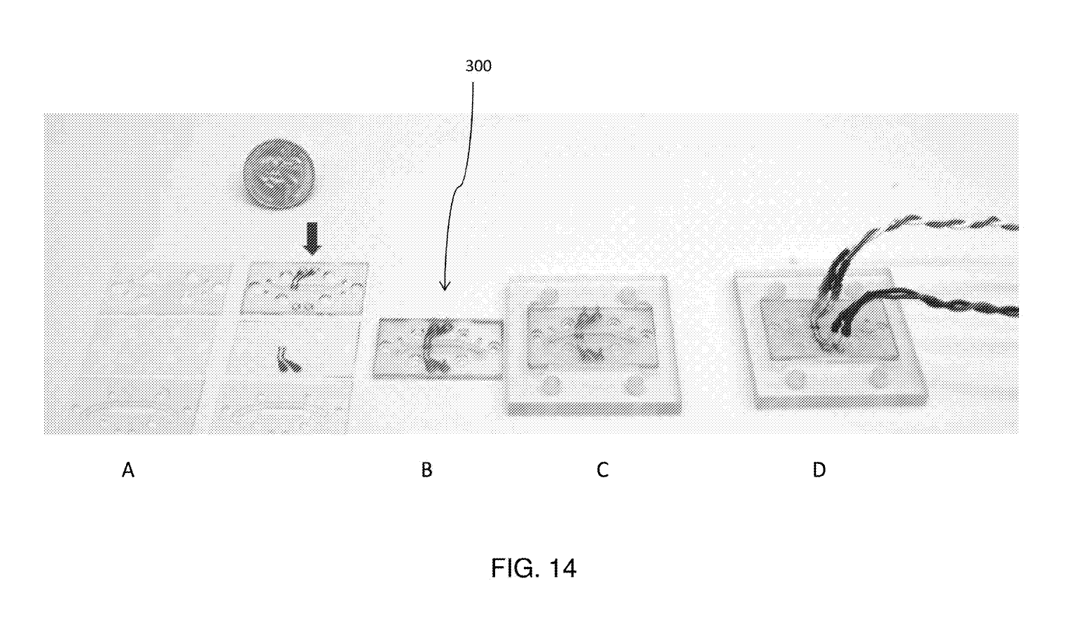

FIG. 11 is a schematic cross-sectional view of a fluidic device 300 taken across a direction of flow and FIG. 12 is a schematic cross-sectional view of the fluidic device 330 taken along a direction of flow. FIGS. 13 and 14 are images of the fluidic device 300. Turning again to FIGS. 11 and 12, the fluidic device 300 includes a solid support structure 320 having a first chamber 330 separated from a second chamber 340 by a porous membrane 310. At least a portion of the first chamber 330 and the porous membrane 310 define a fluid channel 332 having a surface 333 opposite the porous membrane 310. The fluidic device 300 also includes a first electrode 342 disposed in the fluid channel 332 at least partially overlying the porous membrane 310 and a second electrode 344 disposed on the surface 333 of the fluid channel opposite the first electrode 342. The fluidic device 300 also includes a growth promoting layer 314 disposed in the fluid channel 332 and overlying at least a portion of the first electrode 342 and overlying at least a portion of the porous membrane 310. The growth promoting layer 314 is configured to promote adhesion and growth of epithelial cells, endothelial cells, sensory transducer cells, neuronal cells, hormone-secreting/endocrine cells, glial cells and/or adipocytes. In some embodiments, the fluidic device 310 also includes the cells 316 cultured on the growth promoting layer 314.

In some embodiments, the fluidic device 300 also includes a third electrode 346 disposed in the first fluid channel 332 at least partially overlying the porous membrane 310 and a fourth electrode 348 disposed on the surface 333 of the first fluid channel opposite the third electrode 336 (see FIG. 12). The electrodes are configured for measurement of electrical properties such as impedance across the first fluid channel 332 or impedance of the cells 316 attached to the porous membrane 310. In some embodiments the second chamber includes muscle cells, such as a functional muscle tissue 370. In some embodiments, the second chamber includes a flexible substrate 360 that supports the functional muscle tissue 370. In some embodiments, the functional muscle tissue 370 and flexible substrate 360 are in the form of a muscle tissue strip having a cantilever portion that is free to deflect in response to contractive force exerted by the functional muscle tissue 370. In some embodiments, the second chamber 340 includes a plurality of flexible substrates 360 each supporting a functional muscle tissue 370 as shown in FIG. 12.

In some embodiments, the first fluid channel 332 has a proximal end 334a defined near an inflow portion of the first fluid channel and a distal end 334b defined near an outflow portion of the first fluid channel, and wherein the first electrode 342 and the second electrode 344 are disposed at the proximal end 334a or at the distal end 334b of the first fluid channel 332. In some embodiments, the fluidic device 300 has an upstream end and a downstream end and the first electrode 342 and the second electrode 344 are disposed upstream or downstream of the flexible substrate 360. In some embodiments, the third electrode 346 and the fourth electrode 348 are also disposed upstream or downstream of the flexible substrate 360.

In some embodiments, a thickness of electrodes that at least partially underlie the growth promoting layer (e.g., the first electrode 342 and the third electrode 346) is selected to achieve desired electrical properties without interfering with growth of the cells over the electrode. In some embodiments, the first electrode 342 and the third electrode 346 (if included) each have a thickness in a range of about 20 nm to about 400 nm. In some embodiments, the first electrode 342 and the third electrode 346 (if included) each have a thickness in a range of about 20 nm to about 200 nm. In some embodiments, the first electrode 156 and the second electrode 224 include gold.

In some embodiments, the first electrode 342 and the second electrode 344 include an adhesion layer and an overlying layer. In some embodiments the adhesion layer includes titanium. In some embodiments the overlying layer includes gold. In some embodiments, the adhesion layer has a thickness in a range of about 3 nm to about 10 nm.

In some embodiments, the porous membrane 310 and the cells cultured on growth promoting layer 314 act as a vascular-like barrier between channels of the fluidic device 300. In some embodiments, the porous membrane 310 is composed of or includes a polycarbonate material.

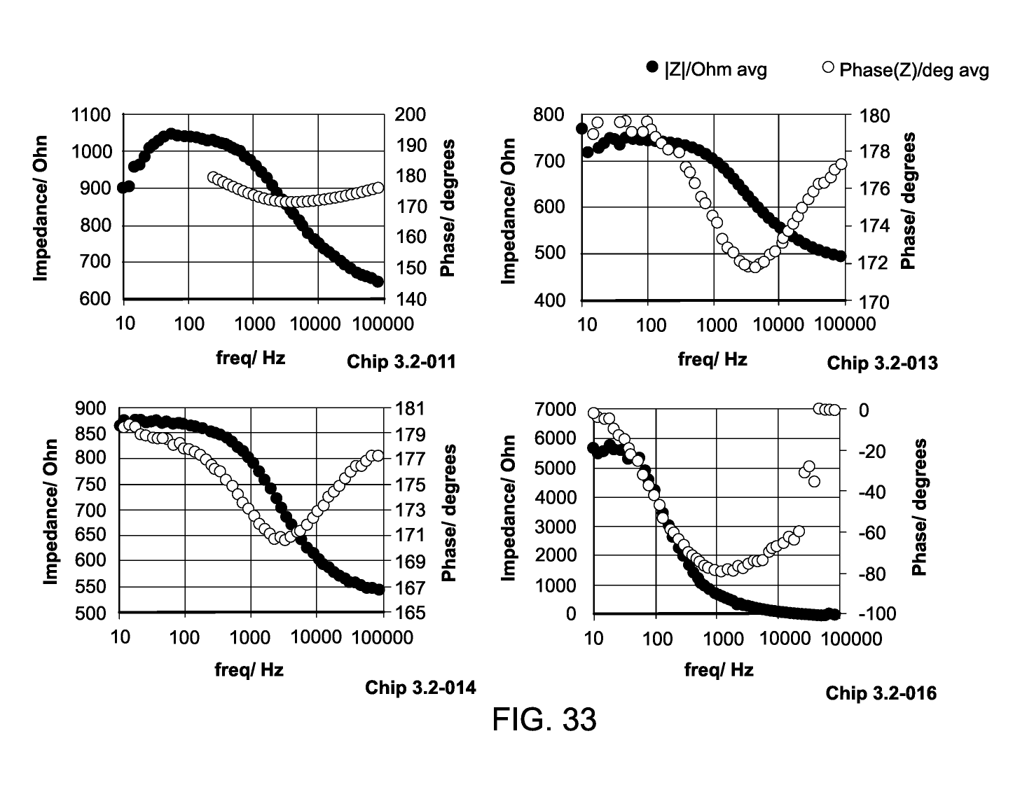

A description of how to obtain impedance measurements using fluidic device 300 is provided below in the examples section below with respect to FIGS. 32 and 33.

Some embodiments include a kit including a fluidic device as described herein (e.g., fluidic device 104, fluidic device 102) and a cell seeding well. FIG. 15 is a perspective view of a cell seeding well 400 according to an embodiment. The cell seeding well 400 includes a well body 410 having a first surface 412 and a second surface bad 414, the well body 410 defining an aperture 416 extending from the first surface 412 to the second surface 414. The shape of the aperture 416 at the second surface corresponds to a shape of the flexible substrate of a fluidic device. The cell seeding well 400 is configured to achieve high densities of cell seeding. For example, in some embodiments, a shape of the aperture 416 tapers from a first cross-sectional area at the first surface 412 to a smaller second cross-sectional area at the second surface 414. This tapering of the aperture 416 produces a funnel effect that can facilitate a high density of cell seeding. The shape of the cell seeding well 410 distributes cells evenly at the bottom of the well 410. The small area of the aperture 416 at the second surface 414 at the bottom of the well enables a high seeding density using a relatively small number of cells. For example, in one embodiment, the well 410 is capable of seeding ten thousand human cardiomyocytes per fluidic device for an aperture 416 having the dimensions of about 5 mm×about 2.5 mm at the second surface 414.

In some embodiments, the seeding well comprises polytetrafluoroethylene (PTFE) (e.g., TEFLON from Chemours Co.), which is non-cytotoxic, autoclavable and easily handled. In some embodiments, the seeding well is machined out of a piece of PTFE.

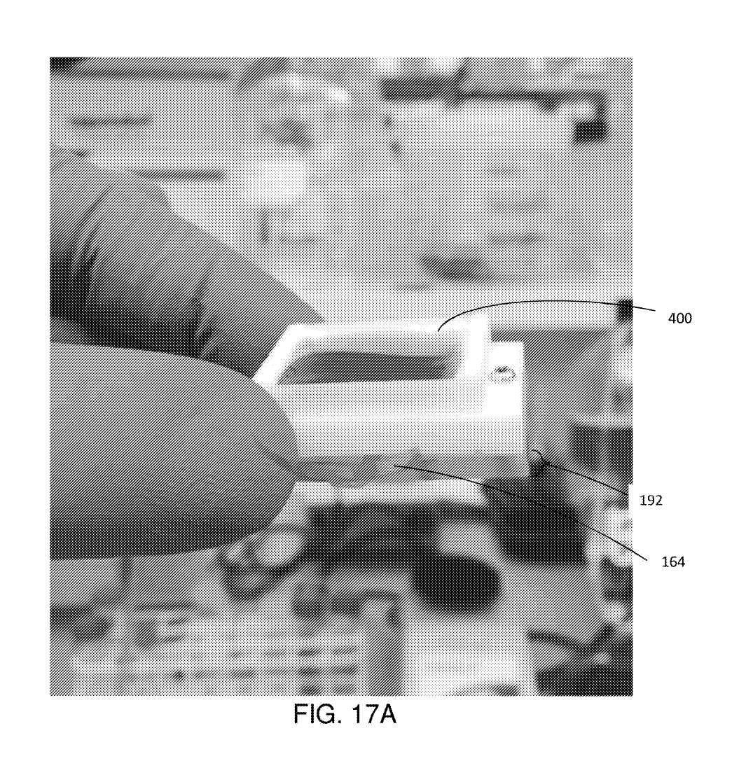

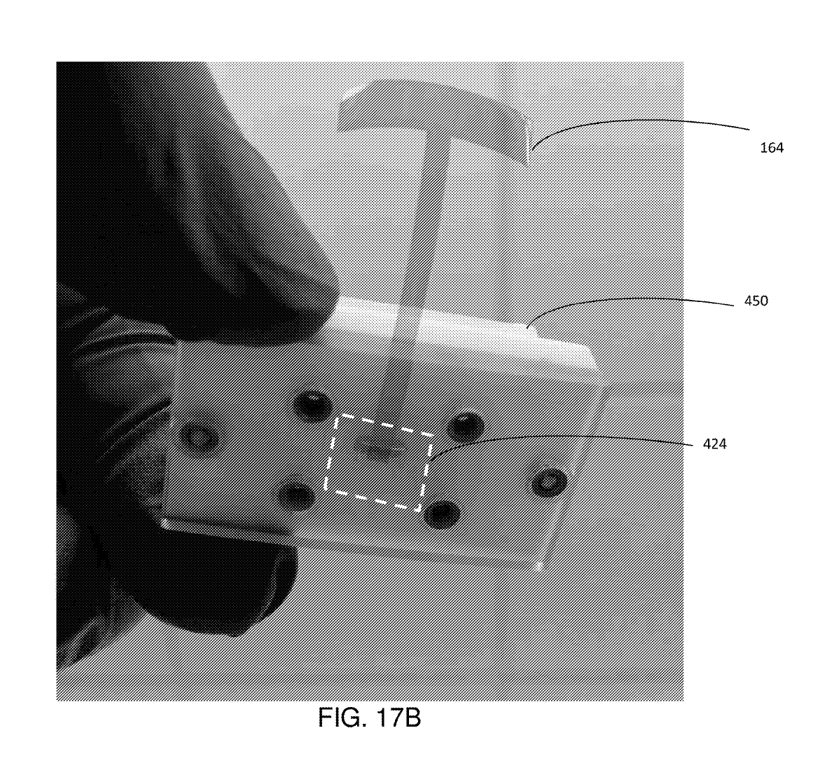

In some embodiments, the cell seeding well 410 is configured to be attached to a second portion 192 of a fluidic device 104 including a base 150, a flexible substrate 160 and a support member 182 when the fluidic device is in a partially disassembled state. For example, cell seeding well 400 has holes 420 through which securing elements (e.g., securing elements 188a) can extend. In some embodiments, a gasket is employed between the seeding well 400 and the base 150. FIG. 16 includes an image of a batch molded set of such gaskets in accordance with an embodiment. In some embodiments, the gasket comprises polydimethylsiloxane (PDMS) (e.g., silicone). FIGS. 17A and 17B are images of a cell seeding well 410 mounted on the second portion 192 of the fluidic device, which includes a flexible electrode array 164 between the flexible substrate and the base 150, being used for cell seeding. In FIG. 17B, dotted line 424 indicates the position of the gasket.

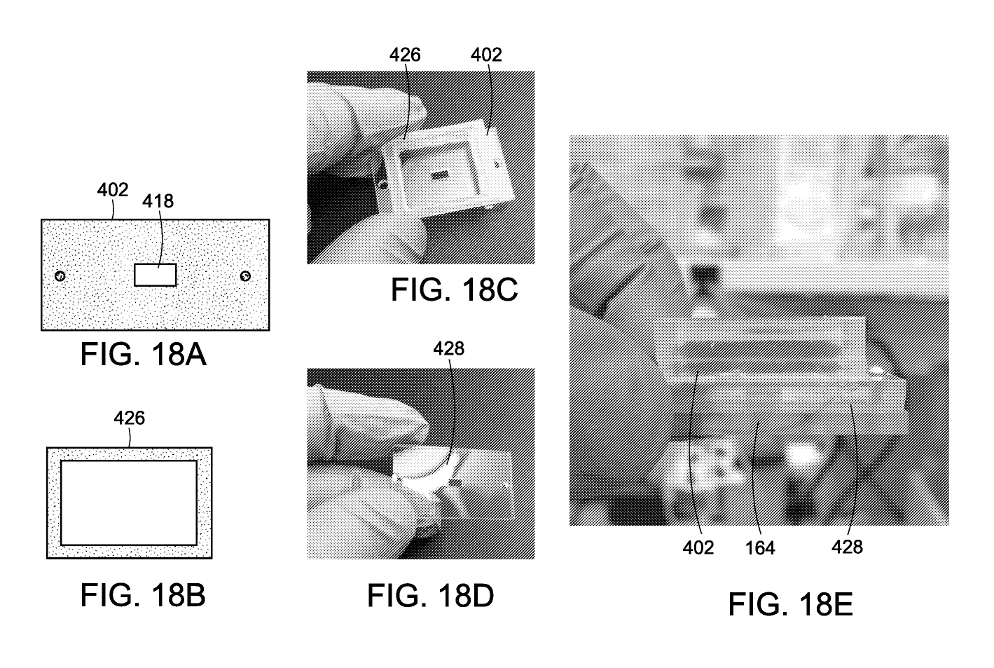

FIGS. 18A through 18E depict another embodiment of a cell seeding system that includes a seeding well 402 (e.g., an acrylic seeding well) defining an aperture 418 corresponding to an area of a flexible substrate, a ring 426 to hold media, and a gasket 428 that may comprise PDMS. FIG. 18C is an image of the ring 426 affixed to the seeding well 402 using epoxy. FIG. 18D is an image of the gasket 428 (e.g., a PDMS gasket), which is configured to be disposed under the seeding well 402. FIG. 18E is an image of the cell seeding well system positioned on the second portion of a fluidic device 164 with the gasket 428 between the second portion of the fluidic device 164 and the seeding well 402.

### Methods of Use of the Devices of the Invention

The devices of the present invention are useful for, among other things, measuring muscle cell activities and functions, investigating muscle developmental biology and disease pathology, drug delivery, as well as in drug discovery and toxicity testing.

To prepare a functional muscle tissue, a flexible substrate comprising a polymer and/or hydrogel layer disposed on the surface of the base of the device is placed in culture with a myocyte suspension, the cells are allowed to settle and adhere to the substrate. In the case of an adhesive surface treatment, cells bind to the flexible substrate in a manner dictated by the micro-scale topological features on a surface of the flexible substrate facing away from the base and the cells respond to the features in terms of maturation, growth and function. The cells on the substrates may be cultured, e.g., in an incubator, under physiologic conditions (e.g., at 37° C.) until the cells form a two-dimensional (2D) tissue (i.e., a layer of cells that is less than about 200 microns thick, or, in particular embodiments, less than about 100 microns thick, less than about 50 microns thick, or even just a monolayer of cells), the anisotropy or isotropy of which is determined by the micro-scale topological features. In one embodiment, the micro-scale topological features are isotropic. In another embodiment, the micro-scale topological features are anisotropic.

Any appropriate cell culture method may be used to establish the tissue on the flexible substrate. The seeding density of the cells will vary depending on the cell size and cell type, but can easily be determined by methods known in the art. In some embodiments, the myocytes are cultured in the presence of, e.g., a fluorophore, nanoparticles and/or fluorescent beads, e.g., fluoro spheres. In one embodiment, a fluorophore, a nanoparticle and/or a fluorescent bead, e.g., a fluoro sphere, is mixed with the gelatin prior to cross-linking and/or the flexible substrate.

The myocytes may be normal myocytes, abnormal myocytes (e.g., those derived from a diseased tissue, or those that are physically or genetically altered to achieve an abnormal or pathological phenotype or function), normal or diseased myocytes derived from embryonic stem cells or induced pluripotent stem cells, or myocytes comprising a genetic construct, such as an expression vector expressing an optogenetic gene, e.g., a light sensitive ion channel (e.g., channelrhodopsin (ChR2), C1V1, Chrimson, Chronos, SSFO, ArchT, ChETA, NpHR, SwiChR, iC1C2, or the like). Myocytes can be cultured in vitro, derived from a natural source, genetically engineered, or produced by any other means. Any natural source of myocytes may be used, including from neonates, e.g., mouse and rat neonates.

Suitable myocytes for the preparation of a functional muscle tissue include, cardiomyocytes, such as ventricular or atrial cardiac cells vascular smooth muscle cells, airway smooth muscle cells, and striated muscle cells, such as skeletal muscle cells, and combinations thereof.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see, e.g., U.S. Pat. Nos. 5,843,780, 6,200,806, the entire contents of each of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, the entire contents of each of which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Furthermore, the term “progenitor cell” is used herein synonymously with “stem cell.”

In one embodiment, progenitor cells suitable for use in the claimed devices and methods are Committed Ventricular Progenitor (CVP) cells as described in PCT Application No. WO 2010/042856, entitled “Tissue Engineered Mycocardium and Methods of Productions and Uses Thereof,” filed Oct. 9, 2009, the entire contents of which are incorporated herein by reference.

Suitable stem cells for use in the present invention are stem cells that will differentiate into a myocyte, the differentiated progeny of such stem cells, and the dedifferentiated progeny of myocytes, and include embryonic (primary and cell lines), fetal (primary and cell lines), adult (primary and cell lines) and iPS (induced pluripotent stem cells). The stem cells may be normal stem cells, abnormal stem cells (e.g., those derived from a diseased tissue, or those that are physically or genetically altered to achieve an abnormal or pathological phenotype or function), normal or diseased cells derived from embryonic stem cells or induced pluripotent stem cells, or cells comprising a genetic construct, such as an expression vector expressing an optogenetic gene, e.g., a light sensitive ion channel (e.g., channelrhodopsin (ChR2), C1V1, Chrimson, Chronos, SSFO, ArchT, ChETA, NpHR, SwiChR, iC1C2, or the like).

Stem cells can be cultured in vitro, derived from a natural source, genetically engineered, or produced by any other means. Any natural source of cells may be used. For example, in one embodiment the stem cells suitable for use in the present invention, e.g., stem cells that give rise to myocytes, may be selected from the group consisting of a primary embryonic stem cell, a stem cell from an embryonic stem cell line, a primary fetal stem cell, a stem cell from a fetal stem cell line, a primary adult stem cell, a stem cell from an adult stem cell line, a stem cell de-differentiated from an adult cell, and an induced pluripotent stem cell (iPS).

In some embodiments, a growth promoting layer is disposed at least partially on a porous membrane and configured to promote adhesion and growth of cells to, e.g., further mimic the in vivo milieu of a functional muscle tissue which includes, among others, blood vessels, nerve cells, fat cells, etc. Thus, the growth promoting layer may be seeded with, for example, epithelial cells, endothelial cells (e.g., vascular endothelial cells), sensory transducer cells, neuronal cells, hormone-secreting/endocrine cells, glial cells, and adipocytes, or combinations thereof.

As discussed above with reference to the seeding of mycoytes, cells may be seeded on a growth promoting layer by placing the growth promoting layer in culture with the cells, allowing the cells to settle and adhere to the growth promoting layer, and culturing the cells, e.g., in an incubator, under physiologic conditions (e.g., at 37° C.) until the cells form a substantially confluent layer.

Any appropriate cell culture method may be used. The seeding density of the cells will vary depending on the cell size and cell type, but can easily be determined by methods known in the art.

The cells seeded on the growth promoting layer may be normal cells, abnormal cells (e.g., those derived from a diseased tissue, or those that are physically or genetically altered to achieve an abnormal or pathological phenotype or function), normal or diseased cells derived from embryonic stem cells or induced pluripotent stem cells, or cells comprising a genetic construct, such as an expression vector expressing an optogenetic gene, e.g., a light sensitive ion channel (e.g., channelrhodopsin (ChR2), C1V1, Chrimson, Chronos, SSFO, ArchT, ChETA, NpHR, SwiChR, iC1C2, or the like). Such cells can be cultured in vitro, derived from a natural source, genetically engineered, or produced by any other means. Any natural source of cells may be used, including from neonates, e.g., mouse and rat neonates.

In some embodiments the devices of the invention include both a functional muscle tissue on a flexible substrate comprising a polymer and/or hydrogel layer disposed on a surface of a base of the device with a functional muscle tissue and cells such as epithelial cells, endothelial cells, sensory transducer cells, neuronal cells, hormone-secreting/endocrine cells, glial cells, and adipocytes, or combinations thereof cultured on a growth a growth promoting layer disposed at least partially on a porous membrane of the device. In such devices, the seeding and culturing of cells to form a functional muscle tissue and the culturing of the cells on the growth promoting layer may be performed on separated portions of the fluidic device prior to assembly of the fluidic device to form the first fluidic channel and the second fluidic channel. Assembling the fluidic device after culturing the functional muscle tissue and the cells on the porous membrane may include positioning the first portion of the device in contact with the second portion of the device and securing the first portion of the device to the second portion of the device using one or more securing elements.

In some embodiments, the fluidic device including functional muscle tissue and cells cultured on the porous membrane is used to determine an electrical property of the epithelial cells, endothelial cells, sensory transducer cells, neuronal cells, hormone-secreting/endocrine cells, glial cells and/or adipocytes and to determine a contractile function of the functional muscle tissue. In some embodiments, the contractile function is a biomechanical activity (e.g., contractility, cell stress, cell swelling, and rigidity).

In some embodiments, the contractile function is an electrophysiological activity such as a voltage parameter (e.g., action potential, action potential duration (APD), conduction velocity (CV), refractory period, wavelength, restitution, bradycardia, tachycardia, and reentrant arrhythmia). In some embodiment the contractile function is a calcium flux parameter (e.g., intracellular calcium transient, transient amplitude, rise time (contraction), decay time (relaxation), total area under the transient (force), restitution, focal and spontaneous calcium release).

In some embodiments, a stimulus (e.g. an electrical stimulus and/or a pharmacological stimulus) is applied before or during measurement of the electrical property of the epithelial cells, endothelial cells, sensory transducer cells, neuronal cells, hormone-secreting/endocrine cells, glial cells and/or adipocytes or before or during measurement of the contractile function of the functional muscle tissue.

Numerous physiologically relevant parameters, e.g., muscle activities, e.g., biomechanical and electrophysiological activities, can be evaluated using the methods and devices of the invention. For example, in one embodiment, the devices of the present invention can be used in contractility assays for contractile cells, such as muscular cells or tissues, such as chemically and/or electrically stimulated contraction of vascular, airway or gut smooth muscle, cardiac muscle, vascular endothelial tissue, or skeletal muscle. In addition, the differential contractility of different muscle cell types to the same stimulus (e.g., pharmacological and/or electrical) can be studied.

In another embodiment, the devices of the present invention can be used for measurements of solid stress due to osmotic swelling of cells. For example, as the cells swell the muscle tissue will contract/bend and as a result, volume changes, force and points of rupture due to cell swelling can be measured.

In another embodiment, the devices of the present invention can be used for pre-stress or residual stress measurements in cells. For example, vascular smooth muscle cell remodeling due to long term contraction in the presence of endothelin-1 can be studied.

Further still, the devices of the present invention can be used to study the loss of rigidity in tissue structure after traumatic injury, e.g., traumatic brain injury. Traumatic stress can be applied to vascular smooth muscle thin films as a model of vasospasm. These devices can be used to determine what forces are necessary to cause vascular smooth muscle to enter a hyper-contracted state. These devices can also be used to test drugs suitable for minimizing vasospasm response or improving post-injury response and returning vascular smooth muscle contractility to normal levels more rapidly.

In other embodiments, the devices of the present invention can be used to study biomechanical responses to paracrine released factors (e.g., vascular smooth muscle dilation due to release of nitric oxide from vascular endothelial cells, or cardiac myocyte dilation due to release of nitric oxide).

In still other embodiments, the devices of the present invention can be used to measure the influence of biomaterials on a biomechanical response. For example, differential contraction of vascular smooth muscle remodeling due to variation in material properties (e.g., stiffness, surface topography, surface chemistry or geometric patterning) of, e.g., a gelatin layer, can be studied.

In further embodiments, the devices of the present invention can be used to study functional differentiation of stem cells (e.g., pluripotent stem cells, multipotent stem cells, induced pluripotent stem cells, and progenitor cells of embryonic, fetal, neonatal, juvenile and adult origin) into contractile phenotypes. For example, undifferentiated cells, e.g., stem cells, are seeded on the devices of the invention and differentiation into a contractile phenotype is observed by observing contraction/bending. Differentiation into an anisotropic tissue may also be observed by quantifying the degree of alignment of sarcomeres and/or quantifying the orientational order parameter (OOP). Differentiation can be observed as a function of: co-culture (e.g., co-culture with differentiated cells), paracrine signaling, pharmacology, electrical stimulation, magnetic stimulation, thermal fluctuation, transfection with specific genes, chemical and/or biomechanical perturbation (e.g., cyclic and/or static strains).

In one embodiment a biomechanical perturbation is stretching of, e.g., the flexible substrate during tissue formation. In one embodiment, the stretching is cyclic stretching. In another embodiment, the stretching is sustained stretching.

The devices of the invention are also useful for evaluating the effects of particular delivery vehicles for therapeutic agents e.g., to compare the effects of the same agent administered via different delivery systems, or simply to assess whether a delivery vehicle itself (e.g., a viral vector or a liposome) is capable of affecting the biological activity of the muscle tissue. These delivery vehicles may be of any form, from conventional pharmaceutical formulations, to gene delivery vehicles. For example, the devices of the invention may be used to compare the therapeutic effect of the same agent administered by two or more different delivery systems (e.g., a depot formulation and a controlled release formulation). The devices and methods of the invention may also be used to investigate whether a particular vehicle may have effects of itself on the tissue. As the use of gene-based therapeutics increases, the safety issues associated with the various possible delivery systems become increasingly important. Thus, the devices of the present invention may be used to investigate the properties of delivery systems for nucleic acid therapeutics, such as naked DNA or RNA, viral vectors (e.g., retroviral or adenoviral vectors), liposomes and the like. Thus, the test compound may be a delivery vehicle of any appropriate type with or without any associated therapeutic agent.

In other embodiments, the devices of the invention can be used to evaluate the effects of a test compound on a contractile function of a functional muscle tissue. Accordingly, in one aspect, the present invention provides methods for identifying a compound that modulates a contractile function of a functional muscle tissue. The methods include providing any one of the devices disclosed herein comprising a functional muscle tissue, e.g., a functional muscle tissue comprising a substantially confluent layer of muscle cells and/or a functional muscle tissue strip, contacting the functional muscle tissue with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound that modulates a contractile function.

In one embodiment, the contractile function is a biomechanical activity, e.g., contractility, cell stress, cell swelling, and/or rigidity. In some embodiment, fluorescent beads are included in the gelatin layer and the biomechanical activity is determined using traction force microscopy.

In some embodiments, e.g., when the device include a functional muscle tissue strip or a plurality of functional muscle tissue strips, determining a biomechanical activity includes determining the degree of contraction, i.e., the degree of curvature or bend of the tissue strip, and the rate or frequency of contraction/rate of relaxation compared to a normal control or control strip in the absence of the test compound (see, e.g., U.S. Pat. No. 9,012,172 and U.S. Patent Publication No. 20140342394, the entire contents of each of which are incorporated herein by reference).

In another embodiment, the contractile function is an electrophysiological activity, e.g., an electrophysiological profile comprising a voltage parameter selected from the group consisting of action potential, action potential morphology, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release, and wave propagation velocity. For example, a decrease in a voltage or calcium flux parameter of a muscle tissue comprising cardiomyocytes upon contraction of the tissue in the presence of a test compound would be an indication that the test compound is cardiotoxic.

In yet another embodiment, the devices of the present invention can be used in pharmacological assays for measuring the effect of a test compound on the stress state of a tissue. For example, the assays may involve determining the effect of a drug on tissue stress and structural remodeling of the muscle tissue. In addition, the assays may involve determining the effect of a drug on cytoskeletal structure (e.g., sarcomere alignment) and, thus, the contractility of the muscle tissue.

In another embodiment, the devices of the invention may be used to determine the toxicity of a test compound by evaluating, e.g., the effect of the compound on an electrophysiological response of a muscle tissue. For example, opening of calcium channels results in influx of calcium ions into the cell, which plays an important role in excitation-contraction coupling in cardiac and skeletal muscle fibers. The reversal potential for calcium is positive, so calcium current is almost always inward, resulting in an action potential plateau in many excitable cells. These channels are the target of therapeutic intervention, e.g., calcium channel blocker sub-type of anti-hypertensive drugs. Candidate drugs may be tested in the electrophysiological characterization assays described herein to identify those compounds that may potentially cause adverse clinical effects, e.g., unacceptable changes in cardiac excitation, that may lead to arrhythmia.

For example, unacceptable changes in cardiac excitation that may lead to arrhythmia include, e.g., blockage of ion channel requisite for normal action potential conduction, e.g., a drug that blocks Na⁺ channel would block the action potential and no upstroke would be visible; a drug that blocks Ca²⁺ channels would prolong repolarization and increase the refractory period; blockage of K⁺ channels would block rapid repolarization, and, thus, would be dominated by slower Ca²⁺ channel mediated repolarization.

In addition, metabolic changes may be assessed to determine whether a test compound is toxic by determining, e.g., whether contacting with a test compound results in a decrease in metabolic activity and/or cell death. For example, detection of metabolic changes may be measured using a variety of detectable label systems such as fluormetric/chrmogenic detection or detection of bioluminescence using, e.g., AlamarBlue fluorescent/chromogenic determination of REDOX activity (Invitrogen), REDOX indicator changes from oxidized (non-fluorescent, blue) state to reduced state (fluorescent, red) in metabolically active cells; Vybrant MTT chromogenic determination of metabolic activity (Invitrogen), water soluble MTT reduced to insoluble formazan in metabolically active cells; and Cyquant NF fluorescent measurement of cellular DNA content (Invitrogen), fluorescent DNA dye enters cell with assistance from permeation agent and binds nuclear chromatin. For bioluminescent assays, the following exemplary reagents may be used: Cell-Titer Glo luciferase-based ATP measurement (Promega), a thermally stable firefly luciferase glows in the presence of soluble ATP released from metabolically active cells.

In another aspect, the present invention provides methods for identifying a compound useful for treating or preventing a muscle disease. The methods include providing any one of the devices disclosed herein comprising a functional muscle tissue, e.g., a functional muscle tissue comprising a substantially confluent layer of muscle cells and/or a functional muscle tissue strip; contacting a plurality of the muscle tissues with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound useful for treating or preventing a muscle disease. For example, by determining a biomechanical activity of the functional muscle tissue in the presence and absence of a test compound, an increase in the degree of contraction or rate of contraction indicates, e.g., that the compound is useful in treatment or amelioration of pathologies associated with myopathies such as muscle weakness or muscular wasting. Such a profile also indicates that the test compound is useful as a vasocontractor. A decrease in the degree of contraction or rate of contraction is an indication that the compound is useful as a vasodilator and as a therapeutic agent for muscle or neuromuscular disorders characterized by excessive contraction or muscle thickening that impairs contractile function.

Compounds evaluated in this manner are useful in treatment or amelioration of the symptoms of muscular and neuromuscular pathologies such as those described below. Muscular Dystrophies include Duchenne Muscular Dystrophy (DMD) (also known as Pseudohypertrophic), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (Also known as Landouzy-Dejerine), Myotonic Dystrophy (MMD) (Also known as Steinert's Disease), Oculopharyngeal Muscular Dystrophy (OPMD), Distal Muscular Dystrophy (DD), and Congenital Muscular Dystrophy (CMD). Motor Neuron Diseases include Amyotrophic Lateral Sclerosis (ALS) (Also known as Lou Gehrig's Disease), Infantile Progressive Spinal Muscular Atrophy (SMA, SMA1 or WH) (also known as SMA Type 1, Werdnig-Hoffman), Intermediate Spinal Muscular Atrophy (SMA or SMA2) (also known as SMA Type 2), Juvenile Spinal Muscular Atrophy (SMA, SMAS or KW) (also known as SMA Type 3, Kugelberg-Welander), Spinal Bulbar Muscular Atrophy (SBMA) (also known as Kennedy's Disease and X-Linked SBMA), Adult Spinal Muscular Atrophy (SMA). Inflammatory Myopathies include Dermatomyositis (PM/DM), Polymyositis (PM/DM), Inclusion Body Myositis (IBM). Neuromuscular junction pathologies include Myasthenia Gravis (MG), Lambert-Eaton Syndrome (LES), and Congenital Myasthenic Syndrome (CMS). Myopathies due to endocrine abnormalities include Hyperthyroid Myopathy (HYPTM), and Hypothyroid Myopathy (HYPOTM). Diseases of peripheral nerves include Charcot-Marie-Tooth Disease (CMT) (Also known as Hereditary Motor and Sensory Neuropathy (HMSN) or Peroneal Muscular Atrophy (PMA)), Dejerine-Sottas Disease (DS) (Also known as CMT Type 3 or Progressive Hypertrophic Interstitial Neuropathy), and Friedreich's Ataxia (FA). Other Myopathies include Myotonia Congenita (MC) (Two forms: Thomsen's and Becker's Disease), Paramyotonia Congenita (PC), Central Core Disease (CCD), Nemaline Myopathy (NM), Myotubular Myopathy (MTM or MM), Periodic Paralysis (PP) (Two forms: Hypokalemic—HYPOP—and Hyperkalemic—HYPP) as well as myopathies associated with HIV/AIDS.

The methods and devices of the present invention are also useful for identifying therapeutic agents suitable for treating or ameliorating the symptoms of metabolic muscle disorders such as Phosphorylase Deficiency (MPD or PYGM) (Also known as McArdle's Disease), Acid Maltase Deficiency (AMD) (Also known as Pompe's Disease), Phosphofructokinase Deficiency (PFKM) (Also known as Tarui's Disease), Debrancher Enzyme Deficiency (DBD) (Also known as Cori's or Forbes' Disease), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD).

In addition to the disorders listed above, the screening methods described herein are useful for identifying agents suitable for reducing vasospasms, heart arrhythmias, and cardiomyopathies.

Vasodilators identified as described above are used to reduce hypertension and compromised muscular function associated with atherosclerotic plaques. Smooth muscle cells associated with atherosclerotic plaques are characterized by an altered cell shape and aberrant contractile function. Such cells are used to prepare a functional muscle tissue on a device of the invention, exposed to candidate compounds as described above, and a contractile function evaluated as described above. Those agents that improve cell shape and function are useful for treating or reducing the symptoms of such disorders.

Smooth muscle cells and/or striated muscle cells line a number of lumen structures in the body, such as uterine tissues, airways, gastrointestinal tissues (e.g., esophagus, intestines) and urinary tissues, e.g., bladder. The function of smooth muscle cells on thin films in the presence and absence of a candidate compound may be evaluated as described above to identify agents that increase or decrease the degree or rate of muscle contraction to treat or reduce the symptoms associated with a pathological degree or rate of contraction. For example, such agents are used to treat gastrointestinal motility disorders, e.g., irritable bowel syndrome, esophageal spasms, achalasia, Hirschsprung's disease, or chronic intestinal pseudo-obstruction.

Any of the screening methods of the invention generally comprise determining the effect of a test compound on a functional muscle tissue as a whole, however, the methods of the invention may comprise further evaluating the effect of a test compound on an individual cell type(s) of the muscle tissue.

In some aspects of the methods of the invention, such as when the devices of the invention include a growth promoting layer disposed at least partially on a porous membrane with cells cultured on the growth promoting layer, (e.g., epithelial cells, endothelial cells, sensory transducer cells, neuronal cells, hormone-secreting/endocrine cells, glial cells, and adipocytes, or combinations thereof) the methods of the invention may include evaluating the health and/or integrity of the cells cultured on the growth promoting layer. In other aspects of the methods of the invention, such as when the devices of the invention include both a functional muscle tissue cultured on a flexible substrate, which includes a polymer and/or hydrogel layer disposed on the surface of the base of the device, and cells (e.g., epithelial cells, endothelial cells, sensory transducer cells, neuronal cells, hormone-secreting/endocrine cells, glial cells, and adipocytes, or combinations thereof) cultured on a growth promoting layer disposed at least partially on a porous membrane, the methods of the invention may further include evaluating the health and/or integrity of the functional muscle tissue.

For example, in one embodiment, an electrical property of the cells on the growth promoting layer may be determined by contacting the cells with a test compound; and determining the effect of the test compound on an electrical property in the presence and absence of the test compound, wherein a modulation of the electrical property in the presence of the test compound as compared to the electrical property in the absence of the test compound indicates that the test compound modulates an electrical property of the cells.

In one embodiment, the electrical property of the cells is impedance of the cells. In one embodiment, the cells on the growth promoting layer are epithelial cells. In another embodiment, the cells on the growth promoting layer are endothelial cells, e.g., vascular endothelial cells.

In some embodiments, the impedance of the cells on the growth promoting layer is determined by methods which include providing data regarding a measured baseline frequency-dependent electrical impedance across the fluid channel of the devices of the invention. The methods include providing a device with a growth promoting layer disposed on a porous membrane of the fluidic device; culturing a layer of cells, e.g., endothelial cells, on the growth promoting layer; stimulating the fluidic device with an electrical current; measuring electrical data from, e.g., a first, second, third, and/or fourth electrodes; and calculating impedance of the cells, e.g., endothelial cells, by subtracting a measured baseline frequency-dependent electrical impedance across the fluid channel from the measured electrical data.

In some embodiment, determining impedance includes measuring current via first and third electrodes, and measuring voltage via second and fourth electrodes.

In some embodiments, providing data regarding the measured baseline frequency-dependent electrical impedance across the fluid channel of the device comprises measuring electrical data from a first, second, third, and fourth electrodes prior to culturing the layer of cells, e.g., endothelial cells, on the growth promoting layer to obtain the measured frequency-dependent baseline electrical impedance across the fluid channel for the fluidic device.

In one embodiment, the fluidic device is simulated with an alternating current of 10 μA.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a functional muscle tissue with a test compound) is intended to include any form of interaction (e.g., direct or indirect interaction) of a test compound and a functional muscle tissue. The term contacting includes incubating a compound and a functional muscle tissue together (e.g., adding the test compound to a functional muscle tissue in culture).

Test compounds, may be any agents including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to a tissue by any suitable means. For example, the test compound may be added drop-wise onto the surface of a device of the invention and allowed to diffuse into or otherwise enter the device, or it can be added to the nutrient medium and allowed to diffuse through the medium. In one embodiment, the screening platform includes a microfluidics handling system to deliver a test compound and simulate exposure of the microvasculature to drug delivery. In one embodiment, the test compound is added to the first fluidic channel comprising a porous membrane and a growth promoting layer comprising cells cultured, e.g., endothelial cells, and the test compound diffuses through the porous membrane in order to contact a functional muscle tissue in a second chamber of the device. In one embodiment, a solution comprising the test compound may also comprise fluorescent particles, and a muscle cell function may be monitored using Particle Image Velocimetry (PIV).

In certain embodiments, the methods of the invention are high throughput methods, where a plurality of test compositions or conditions are screened. For example, in certain embodiments, a library of compositions are screened, where each composition of the library is individually contacted to the co-cultures in order to identify which agents suitable for use as described herein.

In one aspect, any of the methods of the invention may further include applying a stimulus, such as an electrical stimulus or a chemical stimulus, or, in the case of cells expressing an optogenetic gene, a light stimulus, to the cells. In one embodiment, the cells are simulated with an alternating current of 10 μA.

### Examples

### Development of Techniques and Materials for Inhibiting Cell Adhesion to Base

Before developing the method of inhibiting cell adhesion on a portion of the base by modifying the surface energy of the base using laser etching, the inventors explored many different methods for preventing cells from adhering to the base material around the gelatin flexible substrate. The methods and techniques tried including use of an acrylic seeding mask which was removed after seeding to remove the cells that were not on the flexible substrate. The manual placement of the acrylic mask in sterile conditions was difficult and the acrylic floated requiring vacuum grease to adhere the mask to the base material, which was messy and may prevent penetration of sterilizing light. Although the problem of difficulty in placement was solved with a mask having a placement holder, the acrylic mask still required adhesive to stick to the base. The inventors also tried using a polyimide film (specifically, KAPTON tape from E. I. du Pont de Nemours and Company) as a seeding mask to prevent cells from sticking to the base around the flexible substrate. Unfortunately, removal of the KAPTON tape mask after seeding damaged the functional muscle tissues. The inventors also tried using of a laser cut gold foil seeding mask, which did not work because it was brittle and deformed easily causing leaks or misalignment. A machined combined mask and seeding well did not work because the mask alignment was not perfect due to tolerances in the machined mask and misalignment caused the gelatin flexible substrate to be damaged.

Eventually the inventors developed methods described herein which rely on modification of a surface energy of an areas of the base adjacent to the flexible substrate to inhibit cell attachment to the base.

### Development of Material for the Base

The inventors had previously used glass as a base when forming functional muscle tissues on flexible substrates; however, due to some disadvantages of glass (e.g., fragility, difficulty in machining, and the complexity of activating a glass surface to facilitate bonding with the flexible substrate) the inventors explored a variety of materials as candidate materials for the base. The criteria for the base material included machinability, the ability to activate the surface by oxygen plasma treatment, biocompatibility, and optical properties. The inventors determined that a suitable base material should facilitate bonding of selective portions of the gelatin flexible substrate to the base and for embodiments that incorporate commercially available flexible electrode array, should facilitate bonding between the base and the flexible electrode array. The inventors were also interested in materials that could be cut with a laser.

Several initially tested materials were ruled out. For example polymethyl methacrylate (PMMA, acrylic) had unstable surface activation and hence unstable adhesion to the gelatin. Polycarbonate (PC) released a toxic chlorine gas when cut with a carbon dioxide laser, may discolor when cut, and exhibits autofluoresence that may hinder imaging. Polymethuylpentene (e.g., PERMANOX from Thermo Scientific) was a soft material that scratched easily, melted when cut with a carbon dioxide laser, and commercial available sheets appears to be opaque.

Additional materials evaluated for the base included polyester, specifically THERMANOX from Nunc, Inc., TOPAS COC from TOPAS Advanced Polymers, ZENOR COC from ZEON Corp., and polyimide. The table below includes results of experimental evaluations of the various materials.

| | | Micro electrode | |
|---|---|---|---|
| Slide Material | Machinability | array bonding | Gelatin Adhesion |
| THERMANOX | Good | No | Excellent |
| (Polyester) | (No burning/ | | (Cultured |
| | melting, clean | | cells over |
| | cut with CO2 | | days on chip) |
| | laser) | | |
| TOPAS | Good | Good (Can be | Excellent |
| (Cyclo Olefin | (Can be cut w/ | reversibly | (Cultured |
| Copolymer) | CO2 and UV | bonded by | cells max. |
| | laser) | plasma treating) | weeks on chip) |
| ZEONOR | Good | Not determined | Not determined |
| (Cyclo Olefin | (Can be cut w/ | | |
| Polymer) | CO2 laser) | | |
| Polyimide | Poor | No | Not determined |
| | (Edges burn | | (material |
| | with CO2 laser) | | not tested |
| | | | to poor |
| | | | “processability”) |

The inventors determined that COC materials such as TOPAS and ZEONOR were particularly well suited as base materials due to the ability to modify surface energies of the material to inhibit cell attachment using a laser, the ability to modify the surface energy to promote selective attachment and bonding with a flexible electrode array coated in polyimide and with a gelatin flexible substrate using oxygen plasma treatment, and the ability to machine the material using a laser without burning or melting.

Manufacture of Flexible Substrate on Base with Microelectrode Array

The inventors developed a manufacturing method for forming a micropatterned gelatin flexible substrate on a COC base with a micro electrode array (MEA) probe at least partially disposed between the flexible substrate and the base with the MEA bonded to the base and the gelatin flexible substrate bonded to the MEA and the base. The height of the gelatin substrate needed to be precisely controlled to have sufficiently large height to accommodate the micropatterning of the top surface needed for cell adhesion and to have a sufficiently small height to obtain high quality electrical recordings from the MEA.

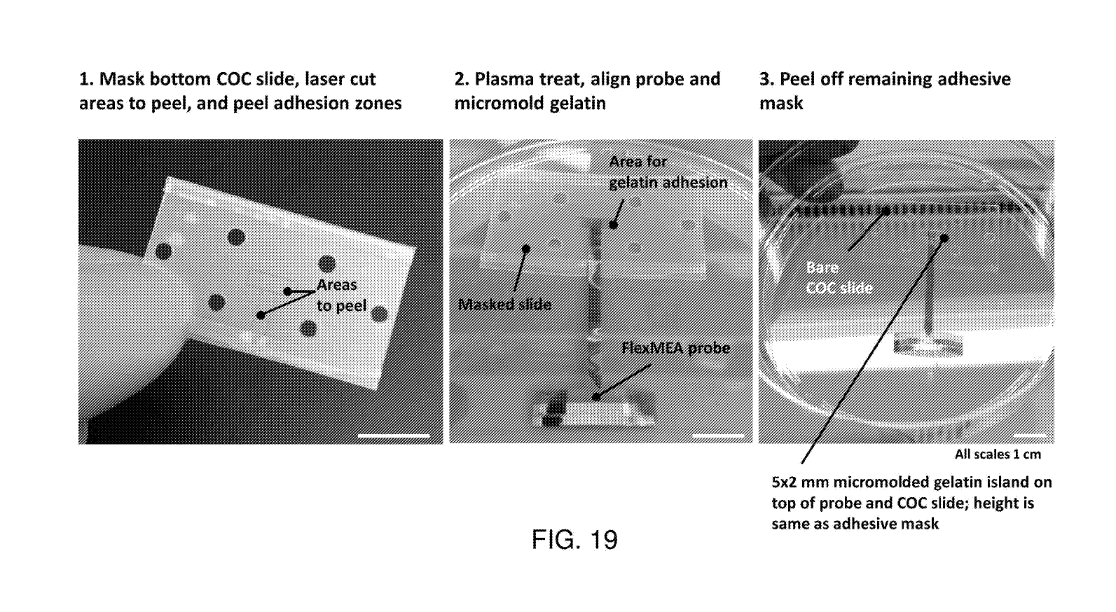

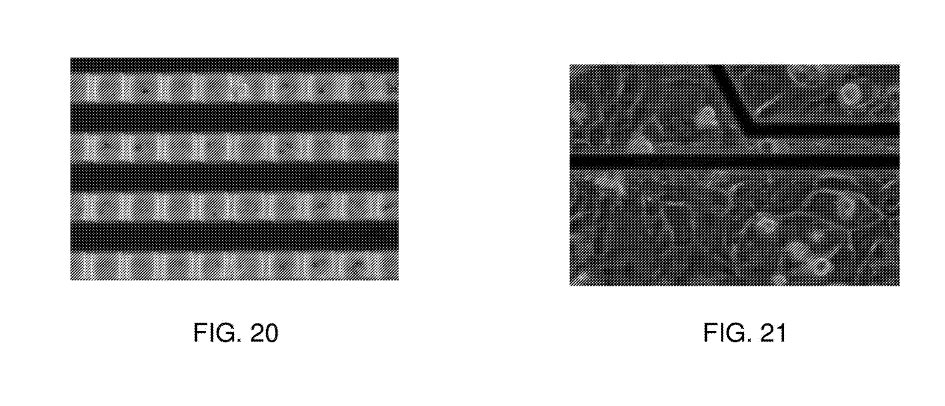

A gelatin flexible substrate was formed on a COC base layer with a microelectrode array disposed at least partially between flexible substrate and the COC base layer. The manufacturing steps are illustrated in FIG. 19. Initially, an adhesive mask was applied to a base in the form of a COC slide and areas to be exposed to oxygen plasma treatment were laser cut and subsequently peeled to expose adhesion zones. The exposed portions of the surface of the base (i.e., the adhesion zones) were plasma treated to modify a surface energy and to promote bonding, and the flexible multiple electrode array (flex MEA) probe was attached to one of the adhesion zone. The gelatin flexible substrate was micromolded on the base over the flex MEA probe and over the other adhesion zone. A polydimethylacrylamide (PDMA) stamp was used to micromold the surface of the gelatin flexible substrate. The adhesive mask acted as a spacer during deposition and of the gelatin such that the height of the gelatin flexible substrate was the thickness of the adhesive mask, which, in this example, was about 54 microns. The adhesive mask was removed leaving the gelatin flexible substrate having a micropatterned surface and the flex MEA probe bonded to the COC base. FIG. 20 is an image of the micromolded surface of the gelatin flexible substrate over the embedded flex MEA probe. FIG. 21 is an image of human cardiomyocytes seeded and cultured 14 days on the gelatin flexible substrate to form a functional muscle tissue over the flex MEA probe.

### Manufacture of Muscle Tissue Strips on a Base

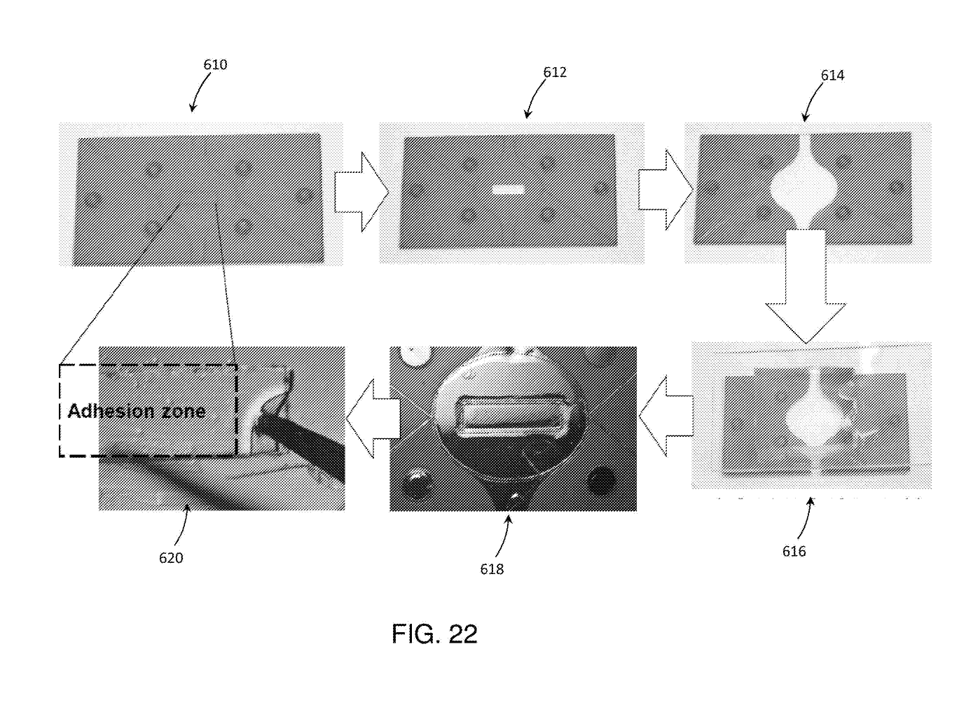

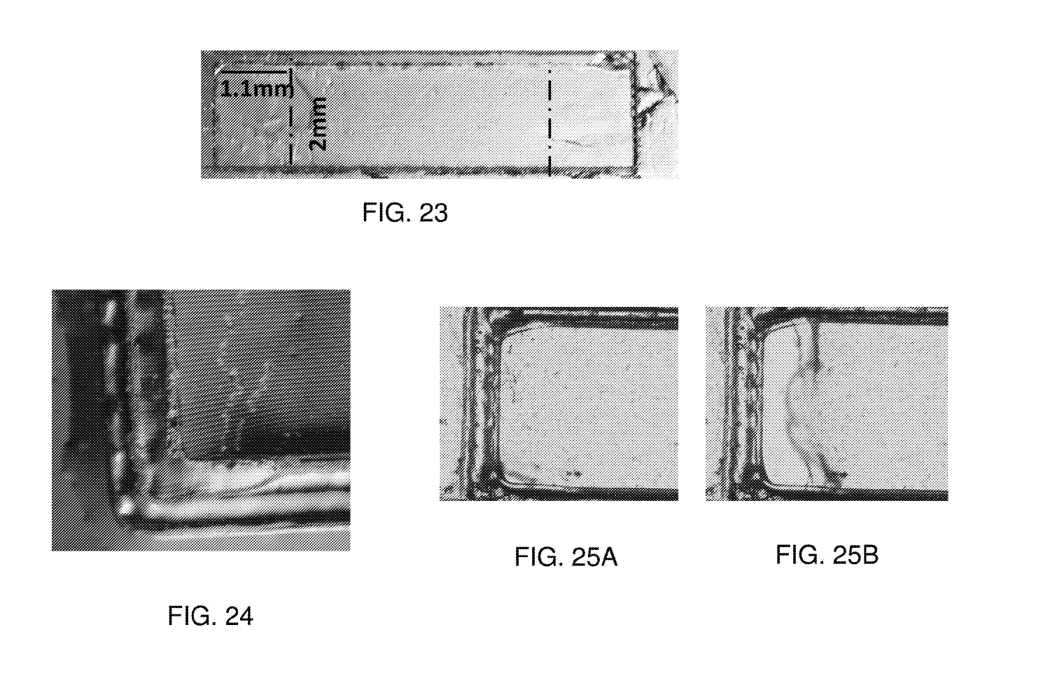

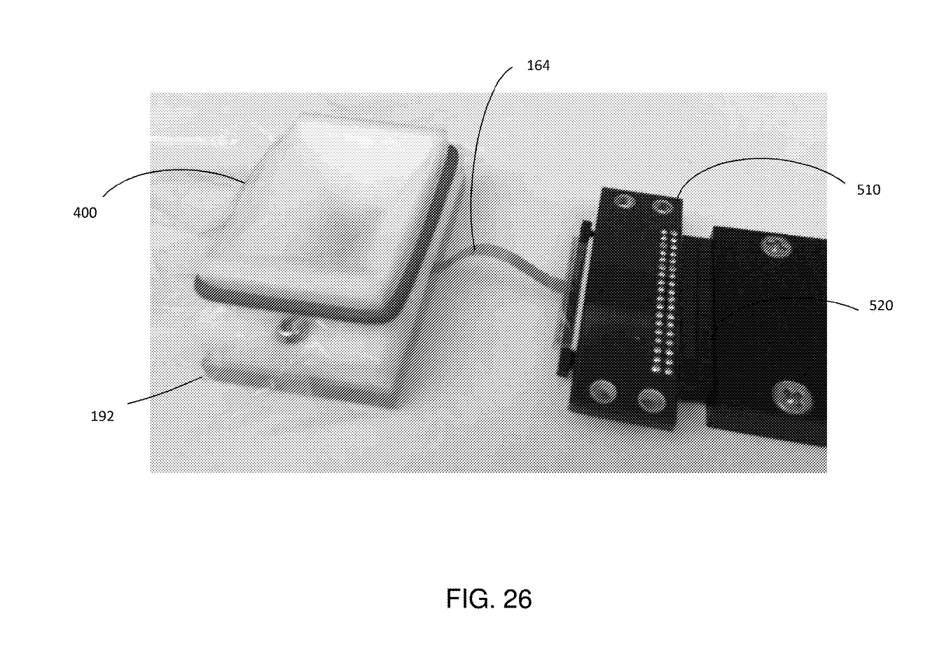

Some methods for forming a functional muscle tissue on an flexible layer having a cantilever portion that deflects away from an underlying base due to contractile forces in the functional muscle tissue require that the cantilever portion be mechanically freed from the underlying base (e.g., through mechanical peeling) prior to use of the device. The inventors developed a method of forming a muscle tissue strip having one or more cantilever portions in which the cantilever portions are free to bend away from the underlying base without having to peel the cantilever portions of the muscle tissue strip away from the underlying base. FIG. 22 illustrates steps in forming a “winged” muscle tissue strip having a cantilevered portion at each end of the strip. Initially, the COC base is masked with an adhesive mask having a cutout that defines an adhesion zone on the surface the base (step 610). The masked base and the exposed adhesion zone are exposed to an oxygen plasma to change the surface energy of the base in the adhesion zone (e.g., to activate the surface of the base in the adhesion zone) (step 612). A portion of the mask is removed to expose a gelatin casting zone (step 614). Gelatin is cast and micromolded on the masked base using a PDMS stamp (step 616). The shape of the gelatin flexible substrate is laser cut from the micromolded gelatin and the COC surface is laser etched around the flexible substrate to prevent cell adhesion (step 618). The metallic-looking shine on the surface of the gelatin in the image for step 618 is merely a lighting artifact. The extra gelatin and adhesive mask are removed leaving the flexible substrate with two cantilever end portions attached to the base (step 620). In the image for step 620, the cantilever end portion of the flexible substrate is being mechanically lifted to show that it is not attached to the underlying base. FIG. 23 is an image of a resulting flexible substrate with dotted and dashed lines indicated the central portion attached to the underlying base and the end cantilever portions. FIG. 24 is detail view of a corner of a resulting flexible substrate showing the etching of the base adjacent to the flexible substrate, which inhibits cell adhesion to the surface of the base. Cells were seeded on the flexible substrate and cultured to form a muscle tissue strip and when the functional muscle tissue was sufficiently developed, contractile forces in the functional muscle tissue caused deflection of the ends of the muscle tissue strip without the muscle tissue strip having been peeled from the underlying base. FIG. 25A is an image of the end of the muscle tissue strip during diastole (relaxed) and FIG. 25B is an image of same end of the muscle tissue strip deflected away from the underlying base during systole (contracted).

### Development of Cell Seeding Well

Prior to the development of the cell seeding wells described herein for seeding of cells on flexible substrates, the inventors explored many other methods and techniques to facilitate high density cell seeding of the flexible substrate with a relatively small amount of wasted cells. Initially, the inventors developed a silicon seeding gasket that sealed to the base and provided openings to place a droplet with cells on the seeding area. The silicon seeding gasket did not work for long term culture because it developed leaks over time and did not work with embodiments that included MEAs due to leakage. The inventors also developed a silicone gasket that was magnetically clamped to the base layer to prevent leakage, however, the magnetic field appeared to be toxic to immature cardiac cells. An O-ring gasket and a seeding chamber clamped with screws for cell seeding provided too small a volume and the O-ring was potentially toxic to the cells over time.

The inventors initially developed the cell seeding well system shown in FIGS. 18A-18E that included a flat cell seeding well 402 of laser-cut acrylic, a ring 426 to hold cells and media attached to the flat cell seeding well 402 using epoxy and a gasket 428 made of PDMS.

The cells seeding well system shown in FIGS. 18A-18E was tested and found to be leak proof when used with a bottom device portion including a flexible electrode array. The inventors further developed the cell seeding well system resulting in the TEFLON cell seeding well 400 with a tapered geometry that improves the density and uniformity of cell seeding and accompanying PDMS gasket as described above with respect to FIGS. 15 to 17B.

### Testing of Functional Muscle Tissue Using Seeding Well and Second Portion of Fluidic Device

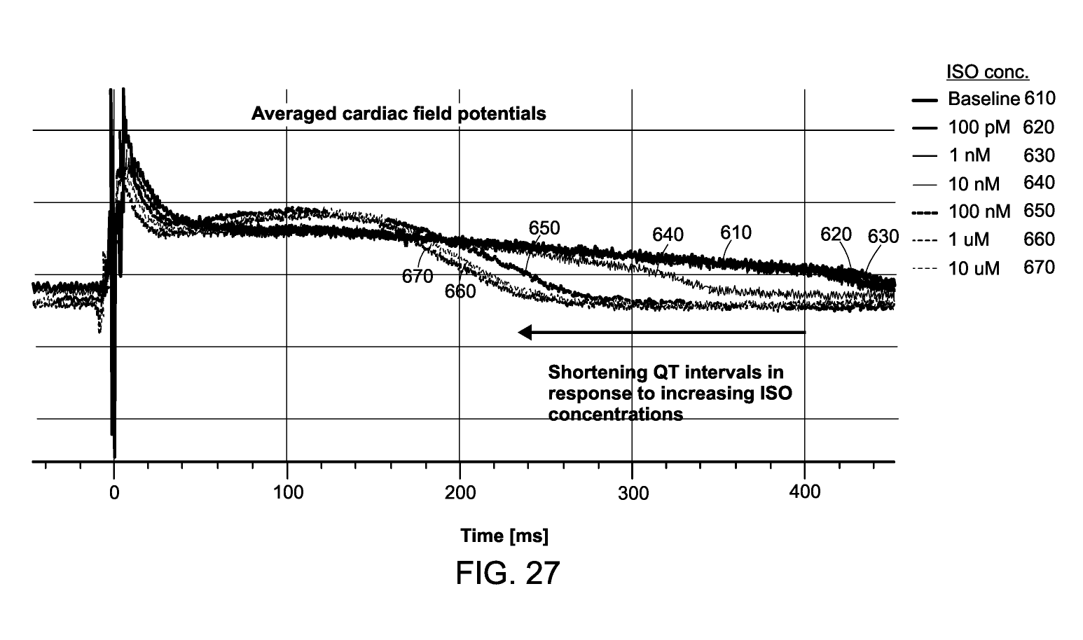

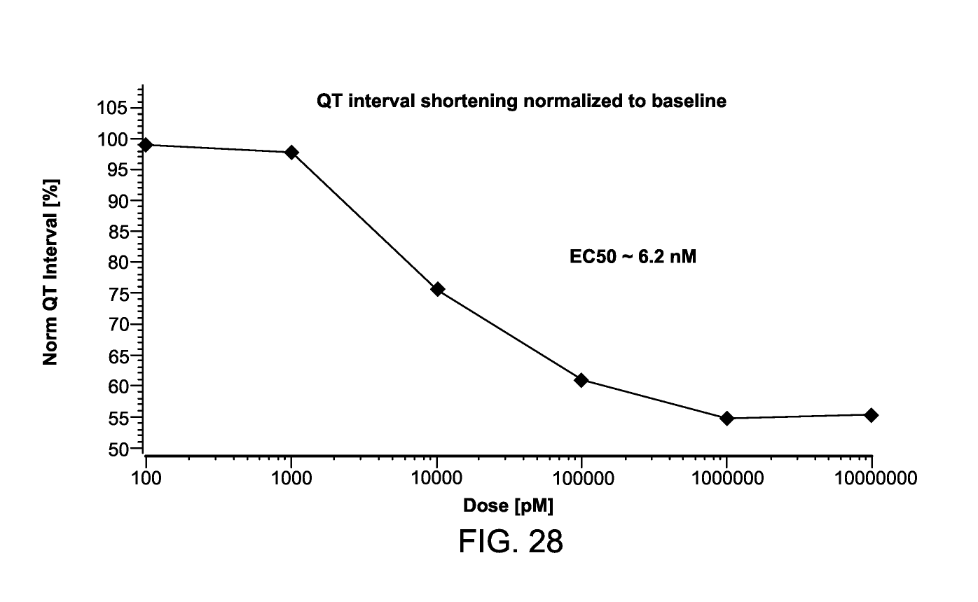

A cell seeding well attached to a second portion of a fluidic device including a gelatin flexible substrate with a micropatterned top surface on a COC base was used to culture a functional muscle tissue of human cardiac tissue on the flexible substrate and to separately evaluate the functional muscle tissue prior to assembling the rest of the fluidic device. FIG. 26 is an image of the cell seeding well 400 attached to the second portion 192 of the fluidic device. The cell seeding well itself is shown in FIGS. 15, 17 and 18. The top of the cell seeding well 400 was wrapped in a gas permeable membrane. A flexible electrode array 164 disposed at least partially between the flexible substrate and the base was used to measure electrical behavior of the cardiac tissue in response to stimulation. The flexible electrode array 164 was attached to an adapter 510 and a preamplifier 520, which were used to obtain electrical signals from the flexible electrode array 164. The cultured functional muscle tissue on the flexible substrate was exposed to escalating doses of isoproterenol, a beta-adrenergic agonist, and resulting cardiac field potentials were measured using the flexible electrode array. FIG. 27 is a graph of the measured cardiac field potentials 610, 620, 330, 640, 650, 660, 670 as a function of time detected by the microelectrode array for various concentrations of isoproterenol. FIG. 28 is a graph of the measured QT interval shortening as a function of the dose of isoproterenol. The shortening QT interval in response to increasing isoproterenol concentrations demonstrates the physiological sensitivity of the lower portion of the fluidic device including the muscle thin film on the flexible substrate and the flexible electrode array. Thus, the modular nature of the fluidic device enabled the flexible substrate on the second portion of the device to be easily seeded and cultured to form the functional muscle tissue, and enabled the functional muscle tissue to be evaluated prior to assembly of the full fluidic device.

### Measurement of Solute Transport Across Endothelial Membrane

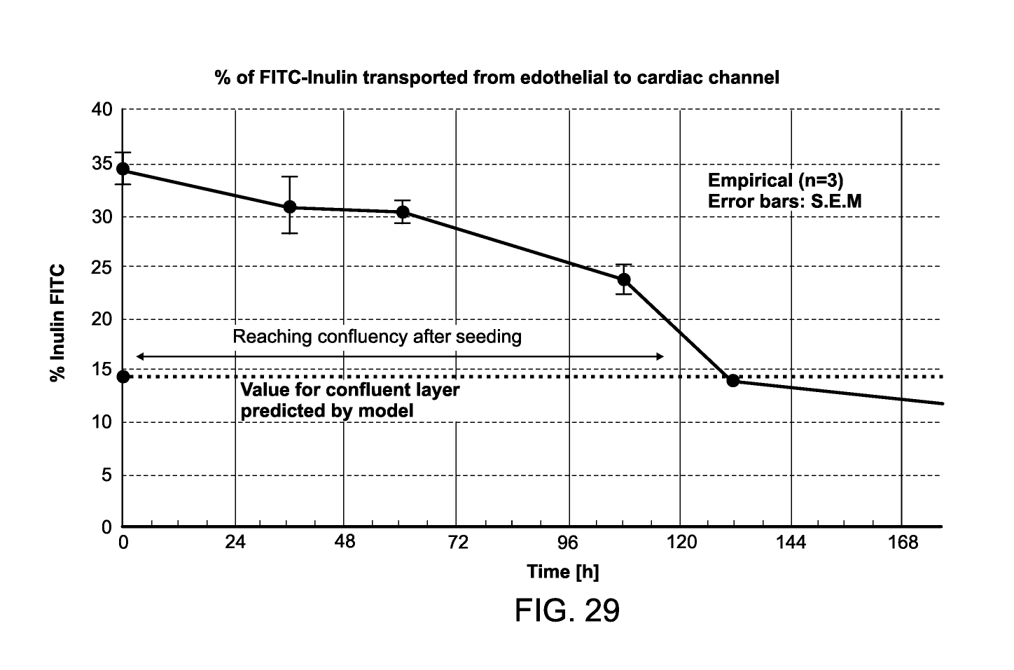

A fluidic device was made that included a porous membrane separating a first fluidic channel from a second fluidic channel with endothelial cells on the porous membrane in the first fluidic channel. Transport of FITC-Inulin across the endothelial cells from the first fluidic channel to the second fluid channel was measured as a function of time after cell seeding with the results shown in a graph in FIG. 29. Transport of FITC-Inulin was established by measuring FITC fluorescence using optical density (OD); because the fluorescent signal strength is proportional to FITC-Inulin concentration, this enabled measurement of the FITC-Inulin concentration in flow inlet and outlet of both channels and hence establish tracer transport across the channels. The measured barrier function of the endothelial cell layer and the porous membrane with regard to solutes was predictable and consistent with computer modeling. After about 130 hours, the measured Inulin-FITC transport rate of 15%, matched the rate predicted for a confluent layer by computer modeling. The expedite data established that the endothelial layer on the porous membrane functioned as a vascular-like barrier to drugs.

### Simulation of Fluid Device Configuration on Drug Uniformity at Functional Muscle Tissue

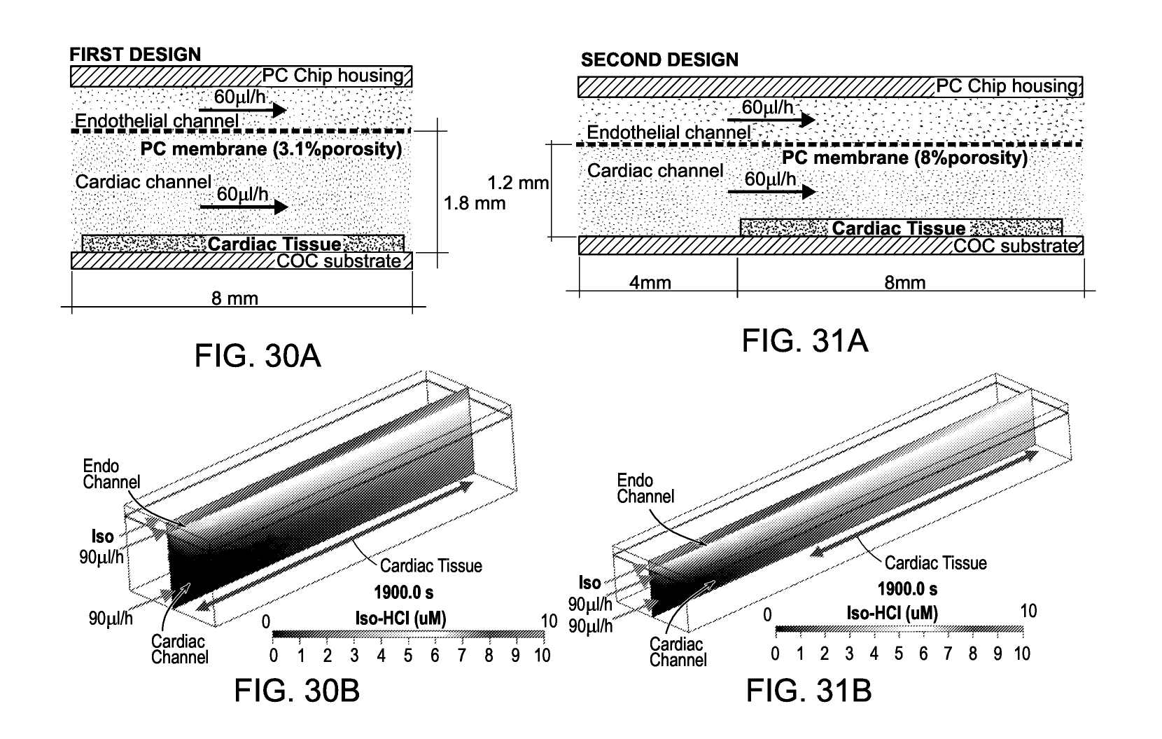

Simulations were performed for fluidic devices having different geometries to improve a uniformity of a drug concentration across the functional muscle tissue. FIG. 30A schematically depicts a first design of a fluidic device. The first design includes a relatively short leading portion between the proximal upstream end of the porous membrane and the portion of the surface of the base covered by the cardiac tissue. The porosity of the porous membrane is 3.1% and the overall device is 8 mm long. FIG. 30B shows results of a simulation of the first fluidic device in which a drug in the first channel diffusing is through an endothelial layer and into the second channel. As shown in FIG. 30B, there is a relatively large gradient in the concentration of the drug along the length of the cardiac tissue.

FIG. 31A schematically depicts a second design of the fluidic device. In the second design, there is a substantial (e.g., 4 mm long) leading portion between the proximal upstream end of the porous membrane and the portion of the surface of the base covered by the cardiac tissue. The height of the second fluidic channel is reduced from 1.8 mm to 1.2 mm and the membrane porosity is increased from 3.1% to 8%. FIG. 31B shows results of a simulation of the second fluidic device in which a drug in the first channel is diffusing through an endothelial layer and into the second channel. As shown in FIG. 31B, there is a relatively smaller gradient in the concentration of the drug along the length of the cardiac tissue as compared to the gradient experience by the cardiac tissue in the simulation of the first design.

### Device for Measurement of Impedance of Cells on Porous Membrane

The inventors made a fluidic device 300 as described above with respect to FIGS. 11 through 14 for obtaining impedance measurements of cells attached to a porous membrane. To obtain impedance measurements using fluidic device 300, alternating current (AC) is applied using an AC frequency sweep and the resulting voltage change as a function of frequency is used to determine the impedance. FIG. 32 identifies how the various electrodes in the fluidic device were used. Specifically the second electrode 344 (the working electrode) and the third electrode 346 (the counter electrode) provided current while the first electrode 342 (the reference electrode) the and the fourth electrode 348 (the sense electrode) were used to measure voltage. Real time impedance measurements were made using a galvanostat 710. A baseline frequency dependent impedance measurement is needed for each fluidic device prior to cell seeding, and then the baseline is subtracted from the measurements taken using the devices after the cells are cultured to obtain the impedance contribution from the cultured cells on the porous membrane. Despite a uniform manufacture process, the baseline frequency dependent impedance appeared to differ for each device requiring that a baseline frequency dependent impedance measurement be obtained for the specific device prior to seeding and culturing of cells on the porous membrane. FIG. 33 includes four graphs of baseline frequency dependent impedance measurements for four different devices showing the variability from device to device.

The techniques, methods and materials disclosed herein for cell seeding and preventing attachment of cells to the base around the flexible substrate and formation of muscle tissue strips without manual peeling enable semi-automated production of fluidic devices including functional muscle tissues.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

As may be recognized by those of ordinary skill in the pertinent art based on the teachings herein, numerous changes and modifications may be made to the above-described and other embodiments of the present disclosure without departing from the spirit of the invention as defined in the appended claims. Accordingly, this detailed description of embodiments is to be taken in an illustrative, as opposed to a limiting, sense. Those skilled in the art will recognize, or be able to ascertain using no more than routine experimentation, many equivalents to the specific embodiments of the described herein. Such equivalents are intended to be encompassed by the following claims.

1.-43. (canceled)

44. A fluidic device comprising:
a porous membrane;
a first channel defining member disposed on the porous membrane, wherein the porous membrane and the first channel defining member define a first fluidic channel;
a support member providing mechanical support for the fluidic device;
a base disposed on the support member;
a second channel defining member disposed on the base, wherein the porous membrane is disposed on the second channel defining member, and wherein the second channel defining member and the porous membrane define a second fluidic channel; and
a flexible substrate comprising a polymer layer and/or a hydrogel layer disposed at least partially on the surface of the base, the flexible substrate configured to support growth of a functional muscle tissue.

45. The fluidic device of claim 106, wherein the fluidic device further comprises a gasket disposed between the base and the second channel defining member.

46. (canceled)

47. The fluidic device of claim 44, further comprising a growth promoting layer disposed on the porous membrane within the first fluidic channel, the growth promoting layer configured to promote adhesion and growth of cells.

48. The fluidic device of claim 44, wherein the base comprises a cyclic olefin copolymer (COC).

49. The fluidic device of claim 44, further comprising a flexible electrode array at least partially disposed between the flexible substrate and the base.

50. The fluidic device of claim 44, further comprising a functional muscle tissue disposed on the flexible substrate.

51. The fluidic device of claim 50, wherein the functional muscle tissue and the flexible substrate form a functional muscle tissue strip having one or two cantilevered portions.

52.-94. (canceled)

95. A device, comprising:
a porous membrane having first and second sides;
endothelial cells disposed on first side of said membrane; and
muscle cells disposed on second side of said membrane.

96. The device of claim 95, wherein said muscle cells comprise cardiac, smooth and/or skeletal muscle cells.

97. The device of claim 95, wherein said muscle cells are part of a muscular thin film.

98. The device of claim 95, wherein said second side comprises at least one electrode.

99. The device of claim 95, wherein said muscle cells are in electrical proximity with at least one electrode.

100. The device of claim 95, wherein said endothelial cells are disposed on a growth promoting layer, said growth promoting layer disposed at least partially on said membrane.

101. A device, comprising:
first and second channels;
endothelial cells disposed in said first channel; and
muscle cells disposed in said second channel.

102. The device of claim 101, wherein said device further comprises a membrane dividing said first and second channels.

103. The device of claim 102, wherein said endothelial cells are disposed on said membrane.

104. The device of claim 102, wherein said muscle cells are disposed on the bottom of said second channel.

105. The device of claim 102, wherein said muscle cells are disposed on a gel layer, said gel layer disposed on the bottom of said second channel.

106. The fluidic device of claim 44, further comprising one or more securing elements that releasably secure the first channel defining member, the porous membrane, the second channel defining member and the base to the support member.

107. A method, comprising:
providing a fluidic device comprising first and second portions;
seeding endothelial cells into said first portion; and
seeding muscle cells into said second portion.

108. The method of claim 107, wherein said muscle cells are seeded on a flexible substrate in second portion.

109. The method of claim 108, wherein the method further comprises culturing the seeded muscle cells to form a functional muscle tissue.

110. The method of claim 107, wherein said endothelial cells are seeded onto a growth promoting layer in said first portion.

111. The method of claim 107, wherein the method further comprises culturing the endothelial cells on the growth promoting layer.
